SEQUENCE LISTING (13 sequences)

SEQ ID NO: 1 - protein, 152 aa, Homo sapiens
MGSSHHHHHHSSGLVPRGSHMCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 2 - protein, 9 aa, Homo sapiens
GTWKLVSSE

SEQ ID NO: 3 - protein, 10 aa, Homo sapiens
FDDYMKEVGV

SEQ ID NO: 4 - protein, 9 aa, Homo sapiens
FATRKVAGM

SEQ ID NO: 5 - protein, 8 aa, Homo sapiens
PNMIISVN

SEQ ID NO: 6 - protein, 7 aa, Homo sapiens
VITIKSE

SEQ ID NO: 7 - protein, 6 aa, Homo sapiens
NTEISF

SEQ ID NO: 8 - protein, 4 aa, Homo sapiens
FDEV

SEQ ID NO: 9 - protein, 9 aa, Homo sapiens
KVKSTITLD

SEQ ID NO: 10 - protein, 8 aa, Homo sapiens
VLVHVQKW

SEQ ID NO: 11 - protein, 10 aa, Homo sapiens
KSTTIKRKRE

SEQ ID NO: 12 - protein, 8 aa, Homo sapiens
KLVVECVM

SEQ ID NO: 13 - protein, 9 aa, Homo sapiens
VTSTRVYER

(19) United States
(12) Patent Application Publication
(10) Pub. No.: US 2004/0219653 A1
(43) Pub. Date: Nov. 4, 2004

(54) Crystal structure of homo sapiens adipocyte lipid binbing protein and uses thereof

(75) Inventor: Xiayang Qiu, Mystic, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/837,378

(22) Filed: Apr. 30, 2004

Related U.S. Application Data
(60) Provisional application No. 60/466,640, filed on Apr. 30, 2003.

(51) Int. Cl. C12N 9/20; G06F 19/00; G01N 33/48; G01N 33/50

(52) U.S. Cl. 435/198; 702/19

(57) ABSTRACT

The invention is directed generally to the structure of lipid binding proteins, particularly human lipid binding protein (aP2), a protein important in adipocyte function. The invention also relates to the use of a crystal structure of human lipid binding protein or mutants and the interaction with ligands for the design of inhibitors. Furthermore, the invention relates to the structure of ligand binding sites.

This application claims the benefit of U.S. Provisional Application No. 60/466,640, filed on Apr. 30, 2003 and incorporated herein by reference in its entirety.

### FIELD OF THE INVENTION

The invention is directed generally to the crystal structure of enzymes. More particularly, the invention relates to the atomic structure of the substrate-binding sites of binding proteins involved in fatty acid sequestration and the use of the structure in drug design.

### BACKGROUND OF THE INVENTION

Intracellular lipid binding proteins have roles in fatty acid solubilization, transfer, and storage in eukaryotic cells. An exemplary intracellular lipid binding protein is adipocyte lipid binding protein (aP2), which binds a variety of fatty acids. In particular aP2 may be essential in lipolysis and lipogenesis, as a carrier of fatty acids between cell membrane and cell organelles. The protein aP2 may thus be important in diseases of lipid and energy metabolism including diabetes.

The murine protein aP2 consists of a monomeric polypeptide of 131 amino acid residues. The murine aP2 has ten anti-parallel β-strands arranged in a barrel structure with an internal ligand binding cavity capable of receiving medium chain and long chain fatty acids and other amphiphilic molecules. Reese-Wagoner et al., Biochim. Biophys. Acta, 1441:106-16 (1999).

An inhibitor of aP2 function has been suggested as useful for treating atherosclerosis. U.S. patent application Publication No. 2002/0035064.

There is an unmet need for methods of identifying modulators of aP2 function that may be useful in treating other lipid and/or metabolic energy diseases and syndromes.

SUMMARY OF THE INVENTION

The invention relates to protein crystal structures and uses thereof in drug design. More particularly, the invention relates to human adipocyte lipid binding protein (aP2) in crystalline form. The invention also relates to a composition comprising the binding protein in crystalline form. The composition can further comprise at least one ligand.

In one embodiment, the invention provides compositions comprising a human lipid binding protein in crystalline form, the binding protein comprising an amino acid sequence at least about 90% homologous to SEQ ID NO:1. In a preferred embodiment of the binding protein the amino acid sequence is at least about 96% homologous to SEQ ID NO:1. In one embodiment the sequence is SEQ ID NO:1.

In a preferred embodiment, the binding protein comprises SEQ ID NO:1 from position 1 to position 152. Shortened variants of SEQ ID NO:1 are also useful in the invention, including sequences lacking up to 21 amino acids from the amino terminal end.

The binding protein can have a first ligand binding site, or a plurality of ligand binding sites. Moreover, the composition comprising the binding protein can comprise at least one ligand. The ligand can be co-crystallized with the binding protein. Suitable ligands include, but are not limited to, palmitate, oleate, bicarbonate, phosphate, and citrate ions. Preferably palmitate or oleate are associated with the first ligand binding site and bicarbonate, phosphate, or citrate ions are associated with another ligand binding site.

In another aspect the lipid binding protein comprises a first ligand binding site defined by at least one amino acid residue selected from the group consisting of Phe³⁷, Tyr⁴⁰, Arg²⁷, Arg⁴⁷, and Tyr⁴⁹. In a preferred embodiment, the crystal can comprise a first ligand binding site defined by amino acid residues 37, 40, 127, 147 and 149 having atoms having atomic coordinates according to FIG. 3.

In yet another aspect, the lipid binding protein comprises a second binding site formed by at least one amino acid residue selected from the group consisting of Phe³⁷ and Tyr⁴⁰. In a preferred embodiment, a part of the ligand binding site is defined by amino acid residues 127, 147, and 149 having atoms having atomic coordinates according to FIG. 3.

One aspect of the invention is directed to methods of designing or identifying a ligand for a lipid binding protein comprising using a three-dimensional structure of a lipid binding protein, employing the three dimensional structure to design or select the ligand, obtaining the ligand, and contacting the ligand with the lipid binding protein to determine binding to the lipid binding protein. One skilled in the art will recognize that the steps of the methods can be carried out in any suitable order based upon the present description. The three-dimensional structure of a binding site can be defined by atomic coordinates of amino acid residues 37, 40, 127, 147 and 149 according to FIG. 3.

The methods can further comprise identifying chemical entities or fragments thereof, capable of binding to the lipid binding protein; and assembling the identified chemical entities or fragments thereof into a single molecule to provide the structure of the ligand.

The ligand can be an inhibitor. In one embodiment the inhibitor is a competitive inhibitor. In another embodiment the inhibitor is a non-competitive inhibitor. The ligand can be designed de novo. Alternatively, the ligand can be designed from a known inhibitor. The methods can further comprise using the atomic coordinates according to FIG. 3, or any suitable portion thereof, of a ligand bound to the lipid binding protein.

Another aspect of the invention is directed to methods for identifying an inhibitor of a mutant lipid binding protein, the methods comprising using a three-dimensional structure of lipid binding protein as defined by atomic coordinates of lipid binding protein according to FIG. 3; replacing one or more lipid binding protein amino acids selected from 37, 40, 54, 59, 74, 96, 97, 127, 138, 147 and 149 of SEQ ID NO:1 in the three-dimensional structure with a different naturally occurring amino acid, thereby forming a mutant lipid binding protein; employing the three-dimensional structure to design or select the inhibitor; synthesizing the inhibitor; and contacting the inhibitor with the mutant lipid binding protein or the lipid binding protein in the presence of a substrate to test the ability of the inhibitor to inhibit the lipid binding protein or the mutant lipid binding protein. The inhibitor can be selected, for example, from a database.

In another aspect, the invention is directed to methods for identifying an inhibitor for a lipid binding protein, comprising using a three-dimensional structure of the binding protein as defined by atomic coordinates of lipid binding protein according to FIG. 3; employing said three-dimensional structure to design or select the inhibitor; synthesizing the inhibitor; and contacting the inhibitor with the binding protein in the presence of a substrate to determine the ability of the inhibitor to inhibit the binding protein.

In one embodiment, the three-dimensional structure can be further defined by atomic coordinates of amino acid residues 106, 126, and 128 according to FIG. 3. In another embodiment, the three-dimensional structure can be further defined by atomic coordinates of amino acid residues 16 and 19, according to FIG. 3.

The ligand can be designed to form a hydrogen bond with at least one amino acid residue selected from the group consisting of Arg²⁷, Arg⁴⁷, and Tyr⁴⁹. In another embodiment, the ligand can be designed to form a hydrophobic bond with at least one amino acid residue selected from the group consisting of Phe³⁷ and Tyr⁴⁰.

The invention also relates to methods of identifying a ligand capable of binding to a lipid binding protein substrate binding site, comprising: (a) introducing into a suitable computer program information defining the binding site comprising first atomic coordinates of amino acids capable of binding to a binding protein substrate, wherein the program displays the three-dimensional structure of the binding site; (b) creating a three dimensional model of a test compound in the computer program; (c) docking the model of the test compound to the structure of the binding site; (d) creating a second three dimensional model of the substrate or an inhibitor of the binding protein and docking the second model thereto; and (e) comparing the docking of the test compound and of the substrate or an inhibitor of the binding protein to provide an output of the program. In one embodiment, the methods further comprise introducing into the computer program second atomic coordinates of water molecules bound to the substrate. In another embodiment, the methods further comprise introducing into the computer program third atomic coordinates of at least one binding protein structural element selected from the group consisting of an alpha helix, a strand of beta sheet, and a coil.

In yet another embodiment the methods further comprise: (f) incorporating the test compound into a biological or biochemical assay for binding protein activity; and (g) determining whether the test compound inhibits binding protein activity in the assay.

The invention is also directed to methods of drug design comprising using the atomic coordinates of a human lipid binding protein, or substantial portion thereof, having at least one ligand binding site, to computationally evaluate relative associations of chemical entities with the ligand binding site.

In another aspect the invention is directed to methods for solving a crystal form comprising using the atomic coordinates of human lipid binding protein crystal, or portions thereof, to solve a crystal form of a mutant, homolog or co-complex of the lipid binding protein by molecular replacement. The methods can further comprise using the atomic coordinates of a ligand bound to lipid binding protein.

One aspect of the invention is directed to machine-readable data storage media comprising a data storage material encoded with machine-readable data comprising atomic coordinates comprising amino acid residues 37, 40, 127, 147, and 149 according to FIG. 3. In another embodiment, the machine-readable data comprise the three-dimensional structure of human lipid binding protein.

In another aspect, the invention comprises computer-implemented tools for design of a drug, comprising: (a) a three-dimensional structure of a lipid binding protein as defined by atomic coordinates of a human lipid binding protein having at least one ligand binding site; (b) a model of a chemical entity; and (c) a computer program addressing the coordinates and capable of modeling the chemical entity in the ligand binding site to produce an output.

In yet another aspect, the invention comprises computers for producing a three-dimensional representation of a lipid binding protein ligand binding site comprising: (a) a machine-readable data storage medium comprising a data storage material encoded with machine-readable data comprising the atomic coordinates comprising the amino acid residues 37, 40, 127, 147, and 149 according to FIG. 3; (b) a working memory for storing instructions for processing the machine-readable data; (c) a central-processing unit coupled to the working memory and to the machine-readable data storage medium for processing the machine readable data into the three-dimensional representation; and (d) a display coupled to the central-processing unit for displaying the three-dimensional representation. The computer can also produce a three-dimensional representation of the ligand binding site of a lipid binding protein; and the machine-readable data can comprise the atomic coordinates of the ligand binding site.

### BRIEF DESCRIPTION OF THE DRAWINGS

FIG. 1 is a ribbon model of the construct including human lipid binding protein with associated ligands.

FIG. 2 is a string model of the construct including human lipid binding protein with associated ligands.

FIG. 3 shows the atomic coordinates of the polypeptide chains of the construct of human lipid binding protein and associated molecules.

DETAILED DESCRIPTION OF THE INVENTION

In order that the invention described herein may be more fully understood, the following detailed description is set forth. The following table lists the amino acid abbreviations used herein.

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

The following terms are used herein as follows, unless stated otherwise:

The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine. Unless specifically indicated, all amino acids referred to in this application are in the L-form.

The term “unnatural amino acids” means amino acids that are not naturally found in proteins. Examples of unnatural amino acids used herein, include selenocysteine and selenomethionine. In addition, unnatural amino acids include D-phenylalanine and the D or L forms of nor-leucine, para-nitrophenylalanine, homophenylalanine, para-fluorophenylalanine, 3-amino-2-benzylpropionic acid, and homoarginine.

The term “positively charged amino acid” includes any naturally occurring or unnatural amino acid having a positively charged side chain under normal physiological conditions. Examples of positively charged naturally occurring amino acids are arginine, lysine and histidine.

The term “negatively charged amino acid” includes any naturally occurring or unnatural amino acid having a negatively charged side chain under normal physiological conditions. Examples of negatively charged naturally occurring amino acids are aspartic acid and glutamic acid.

The term “hydrophobic amino acid” means any amino acid having an uncharged, nonpolar side chain that is relatively insoluble in water. Examples of naturally occurring hydrophobic amino acids are alanine, leucine, isoleucine, valine, proline, phenylalanine, tryptophan and methionine. Histidine and tyrosine can also participate in hydrophobic bonds.

The term “hydrophilic amino acid” means any amino acid having an uncharged, polar side chain that is relatively soluble in water. Examples of naturally occurring hydrophilic amino acids are serine, threonine, tyrosine, asparagine, glutamine, and cysteine.

The term “hydrogen bond” is used to describe an interaction between polar atoms including N, O, and S, in which hydrogen forms a bridge. The side chains of ionic and hydrophilic amino acids and of amide moieties in the peptide backbone are candidates for hydrogen bonds. Polar and ionic moieties in substrates and inhibitors are candidates for hydrogen bonding.

The term “hydrophobic bond” is used to describe a Van der Waals interaction of non-polar moieties that are enthalpicly or entropicly favored over interaction with water or polar groups. Thus, one model for hydrophobic bonds is the gain in free energy formed by exclusion of water. Prime candidates for forming hydrophobic bonds are the aliphatic tail of fatty acids and side chains of amino acid residues including phenylalanine, tryptophan, proline, leucine, isoleucine, valine, alanine, histidine, and tyrosine.

The term “residue” when used in connection with amino acids refers to the part of an amino acid incorporated into a polypeptide.

The term “ligand” refers to a chemical entity that binds to, or associates with, a binding protein. Often, but not always, a ligand is a small molecule. A substrate is a ligand that can be, under appropriate conditions, chemically acted upon by the binding protein. In particular, palmitate is a fatty acid that binds to the binding protein, but does not undergo a chemical reaction.

The term “mutant” refers to a lipid binding protein polypeptide, i.e. a polypeptide displaying the biological activity of wild-type, lipid binding protein, characterized by the replacement of at least one amino acid from the wild-type, lipid binding protein sequence and/or deletion or addition of amino acid sequences, e.g., to SEQ ID NO:1. Such a mutant may be prepared, for example, by expression of lipid binding protein cDNA previously altered in its coding sequence by oligonucleotide-directed mutagenesis, or by using other means well-known in the art.

Lipid binding protein mutants may also be generated by site-specific incorporation of unnatural amino acids into lipid binding protein proteins using, for example, the general biosynthetic method of Noren et al., Science, 244:182-88 (1989). In this method, the codon encoding the amino acid of interest in wild-type lipid binding protein is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant lipid binding protein enzyme with the site-specific incorporated unnatural amino acid.

Selenocysteine or selenomethionine may be incorporated into wild-type or mutant lipid binding protein as described below. In this method, the wild-type or mutagenized lipid binding protein cDNA may be expressed in a host organism on a growth medium depleted of either natural cysteine or methionine (or both) but enriched in selenocysteine or selenomethionine (or both).

Altered surface charge describes a change in one or more of the charge units of a mutant polypeptide, at physiological pH, as compared to wild-type lipid binding protein. This is preferably achieved by mutation of at least one amino acid of wild-type lipid binding protein to an amino acid comprising a side chain with a different charge at physiological pH than the original wild-type side chain.

The change in surface charge is determined, for example, by measuring the isoelectric point (pI) of the polypeptide molecule containing the substituted amino acid and comparing it to the isoelectric point of the wild-type lipid binding protein molecule.

A “competitive” inhibitor is one that inhibits human lipid binding protein activity by binding to the same form of human lipid binding protein as its substrate binds—thus directly competing with the substrate for the active site of human lipid binding protein. Competitive inhibition can be reversed completely by sufficiently increasing the substrate concentration.

An “uncompetitive” inhibitor is one that inhibits human lipid binding protein by binding to a different form of the enzyme than does the substrate. Such inhibitors bind to human lipid binding protein already bound with the substrate and not to the free enzyme. Uncompetitive inhibition cannot be reversed completely by increasing the substrate concentration.

A “non-competitive” inhibitor is one that can bind to either the free or substrate bound form of human lipid binding protein.

Those of skill in the art will appreciate that they can identify inhibitors as competitive, uncompetitive or non-competitive by computer fitting enzyme kinetic data using standard equations, for example, according to Segel, I. H., Enzyme Kinetics, J. Wiley & Sons, (1975).

The term “homologue” as used herein means a protein, polypeptide, oligopeptide, or portion thereof, having preferably at least 90% amino acid sequence identity with human lipid binding protein or any functional or structural domain of lipid binding protein.

The term “co-complex” means lipid binding protein or a mutant or homologue of lipid binding protein in covalent or non-covalent association with a chemical entity or compound.

The term “associating with” refers to a condition of proximity between a chemical entity or compound, or portions thereof, and a lipid binding protein molecule or portions thereof. The association may be non-covalent, wherein the juxtaposition is energetically favored by hydrogen bonding or van der Waals or electrostatic interactions, or it may be covalent.

The terms “beta sheet or β-sheet” refers to the conformation of a polypeptide chain stretched into an extended zig-zag conformation. Portions of polypeptide chains termed strands that run “parallel” all run in the same direction, amino terminus to carboxy terminus. Polypeptide chains or portions thereof, termed “strands”, that are “antiparallel” run in the opposite directions.

The term “binding site” refers to a region of the binding protein comprised of amino acid residues and optionally cofactors to which a ligand can bind. Lipid binding protein has binding sites for at least a fatty acid or other ligand, bicarbonate ion, and phosphate ion.

The term “atomic coordinates” refers to mathematical coordinates derived from mathematical equations related to the patterns obtained on diffraction of a monochromatic beam of X-rays by the atoms (scattering centers) of a lipid binding protein molecule in crystal form. The diffraction data are used to calculate an electron density map of the repeating unit of the crystal. The electron density maps are used to establish the positions of the individual atoms within the unit cell of the crystal. The similar term “structure coordinates” refers to the mathematical coordinates of the individual atoms. It is to be understood that a set of atomic coordinates includes not just the exact coordinates as listed, but any translational or rotational variation in those coordinates, as long as the relative positions of the atoms is maintained.

The term “substantial portion” of atomic coordinates refers to a plurality of at least twelve atomic coordinates that define or partially define the location of several atoms in the binding protein or ligand. Preferably, a substantial portion is at least 24 coordinates. More preferably, a substantial portion is at least 36 coordinates. The coordinates can be within the standard deviation.

The term “heavy atom derivatization” refers to a method of producing a chemically modified form of a crystal of lipid binding protein. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the enzyme. See, for example, Blundel and Johnson, Protein Crystallography, Academic Press (1976).

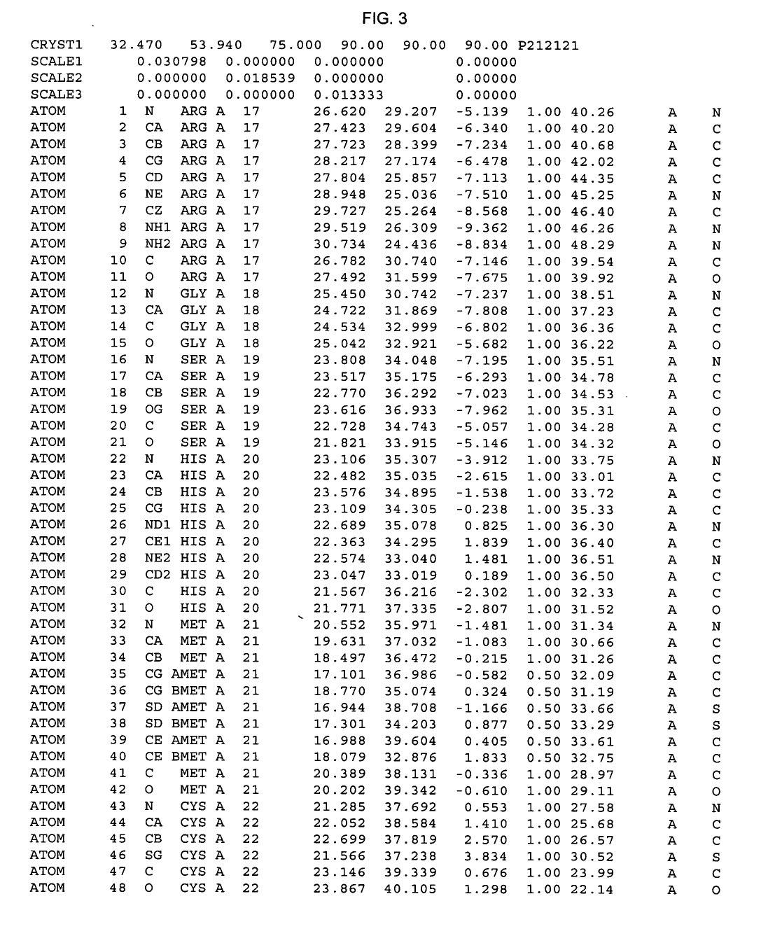

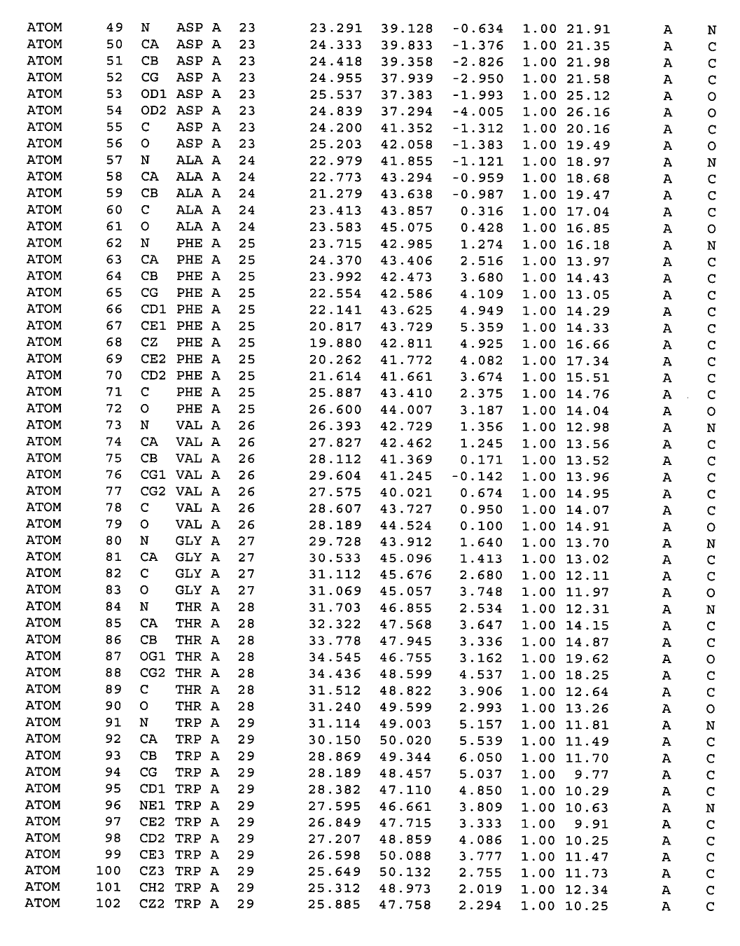

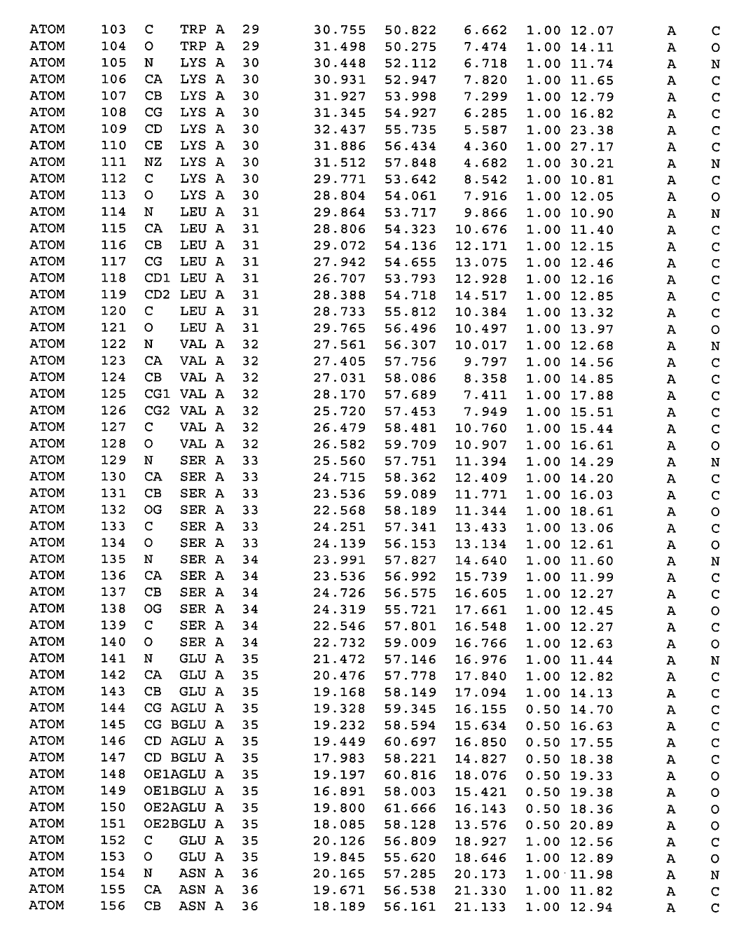

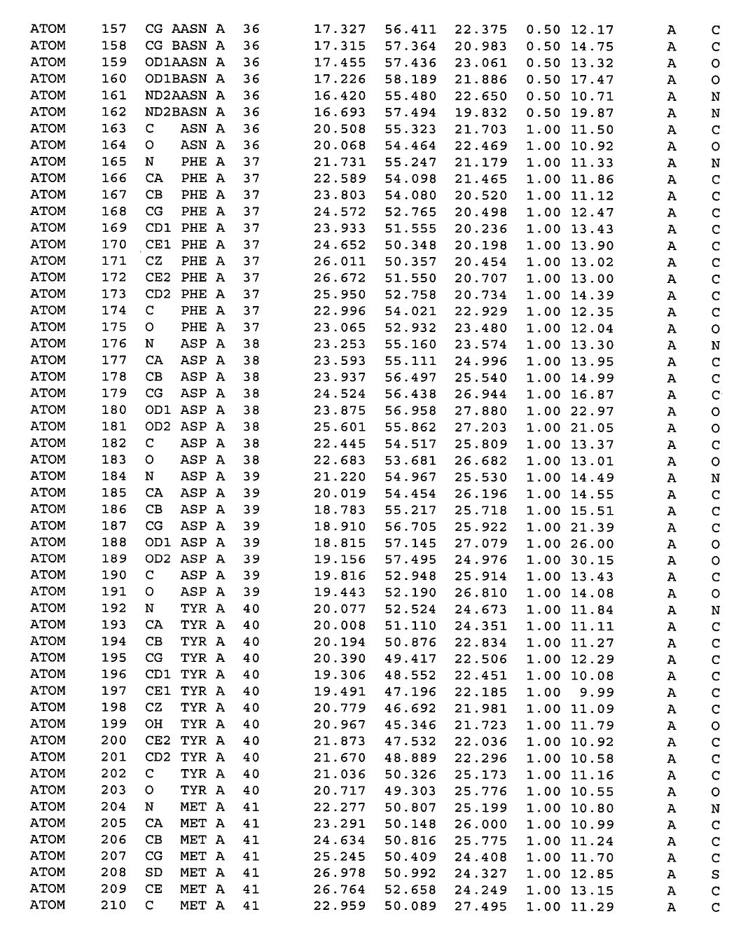

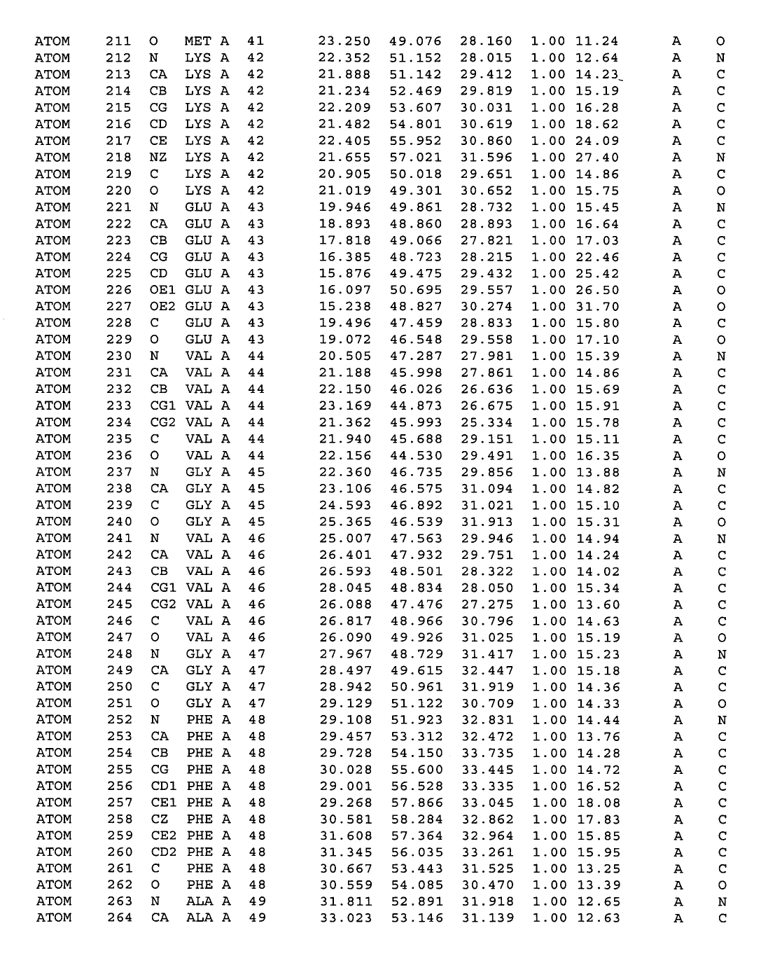

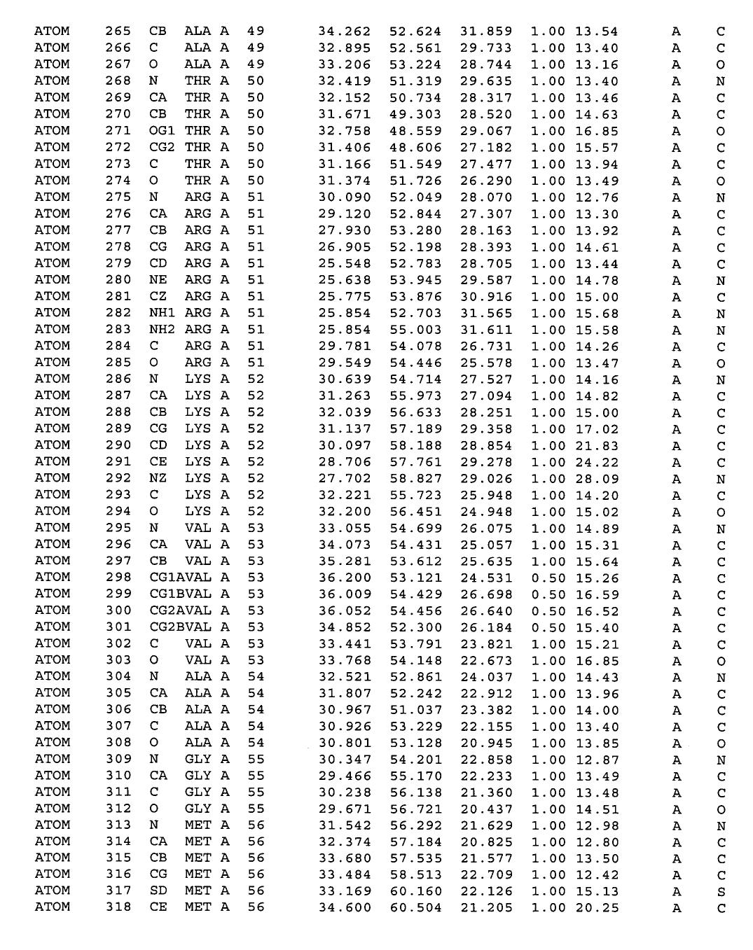

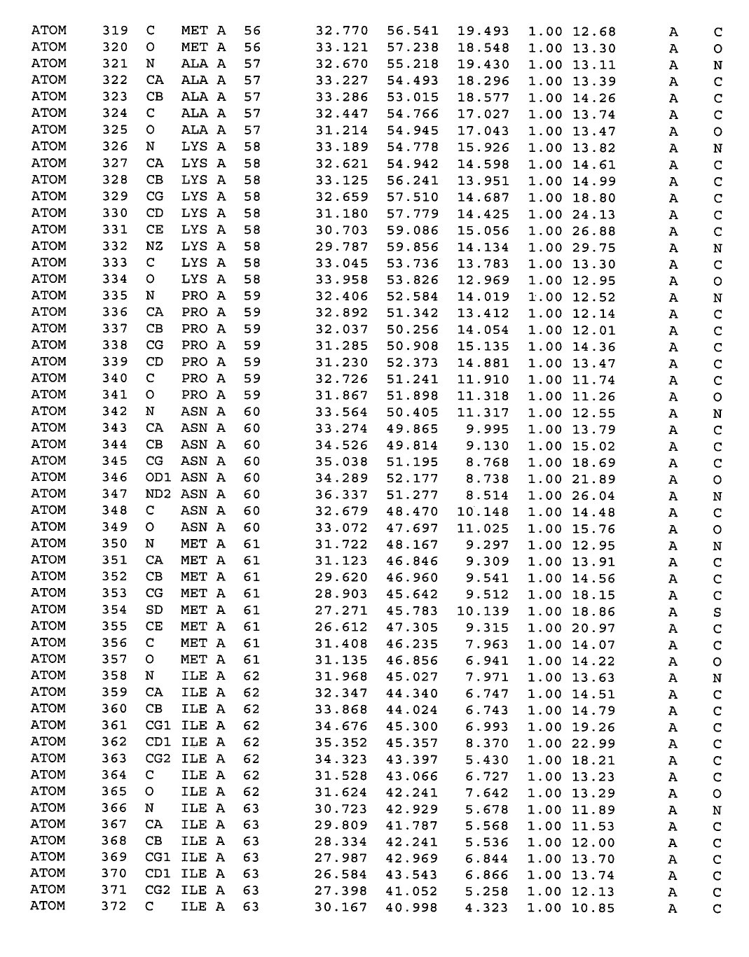

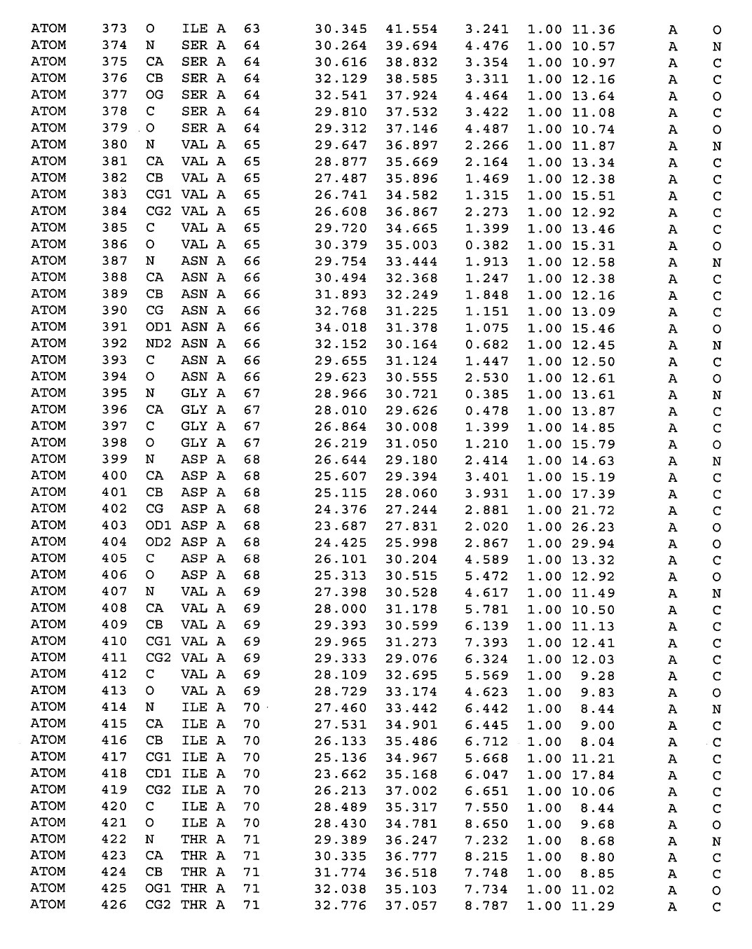

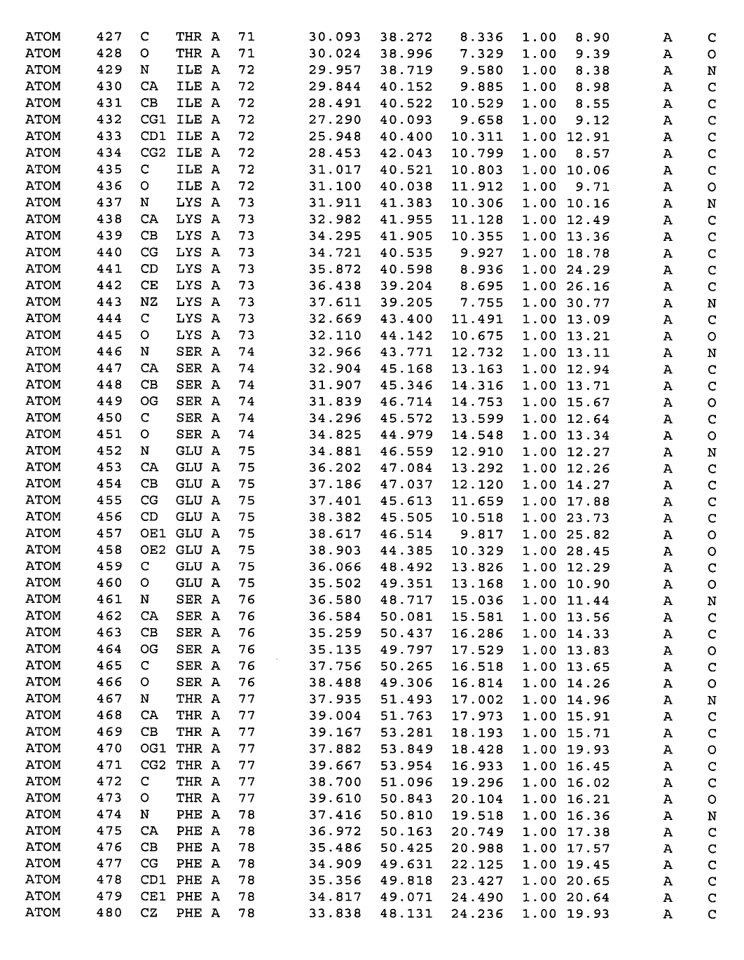

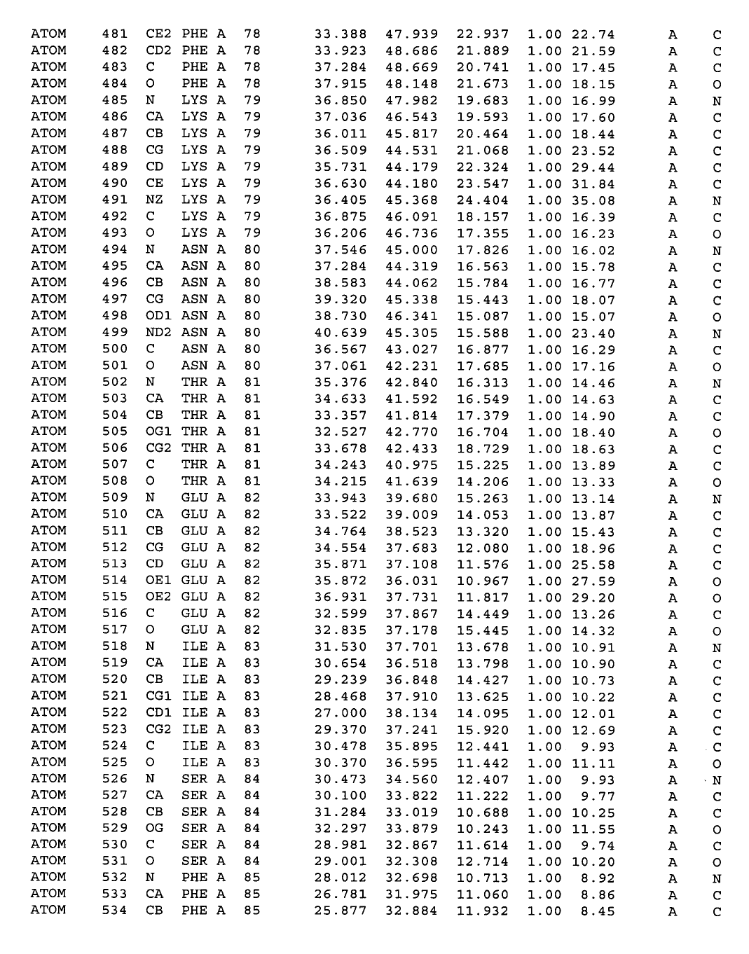

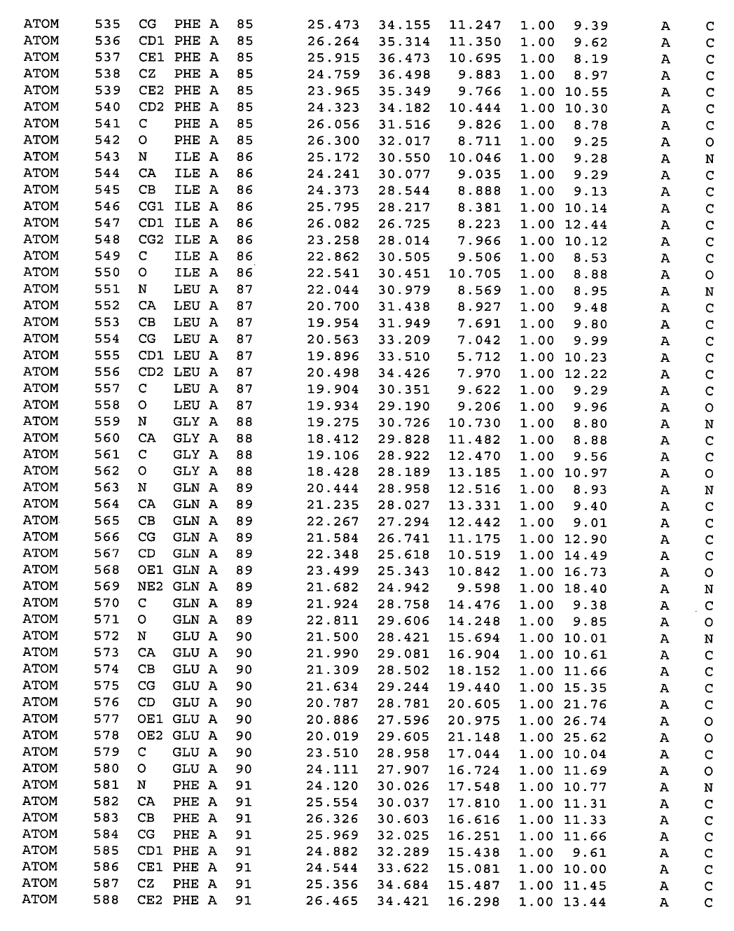

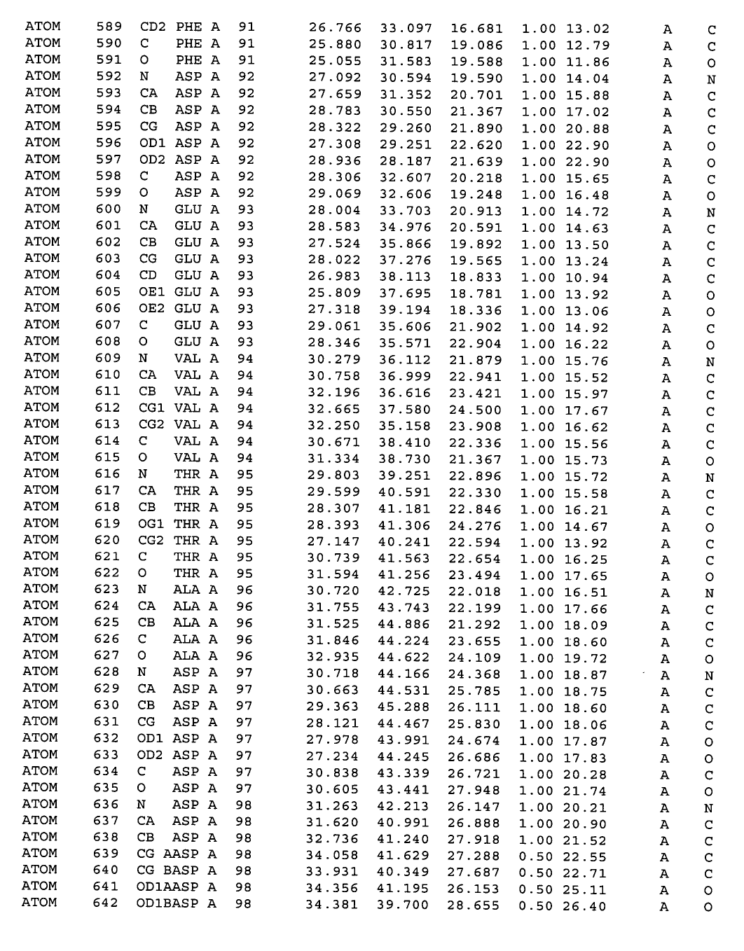

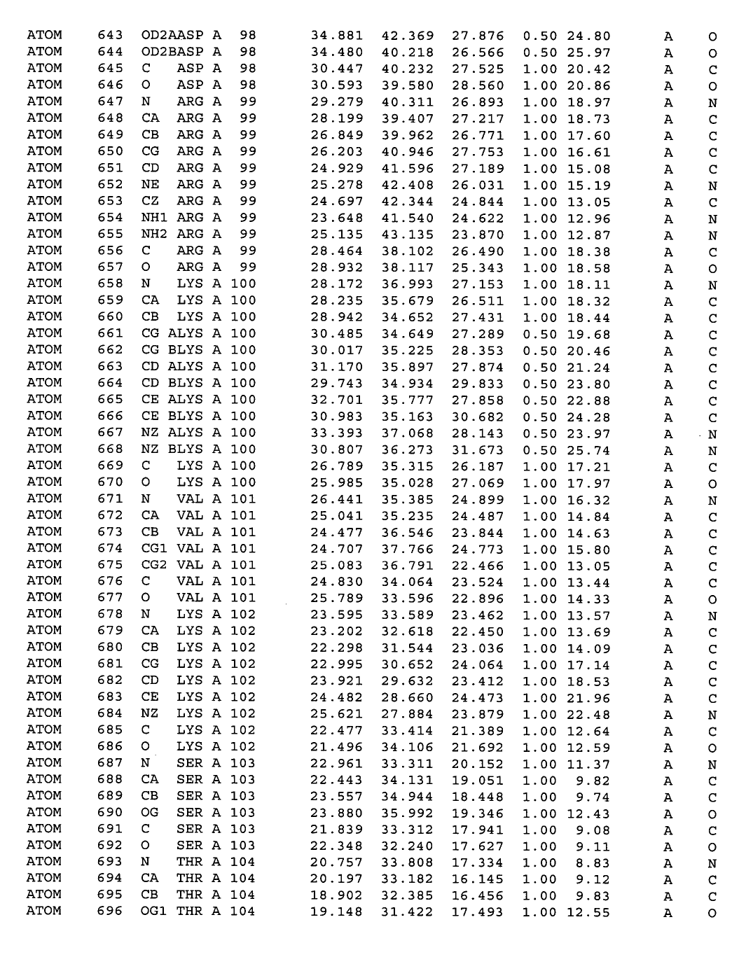

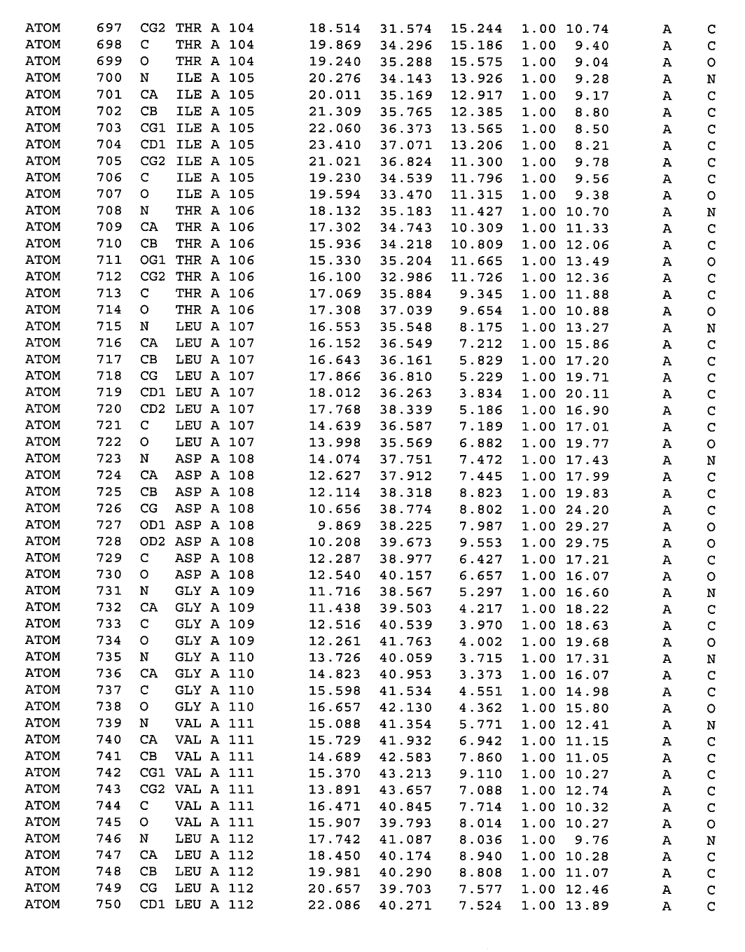

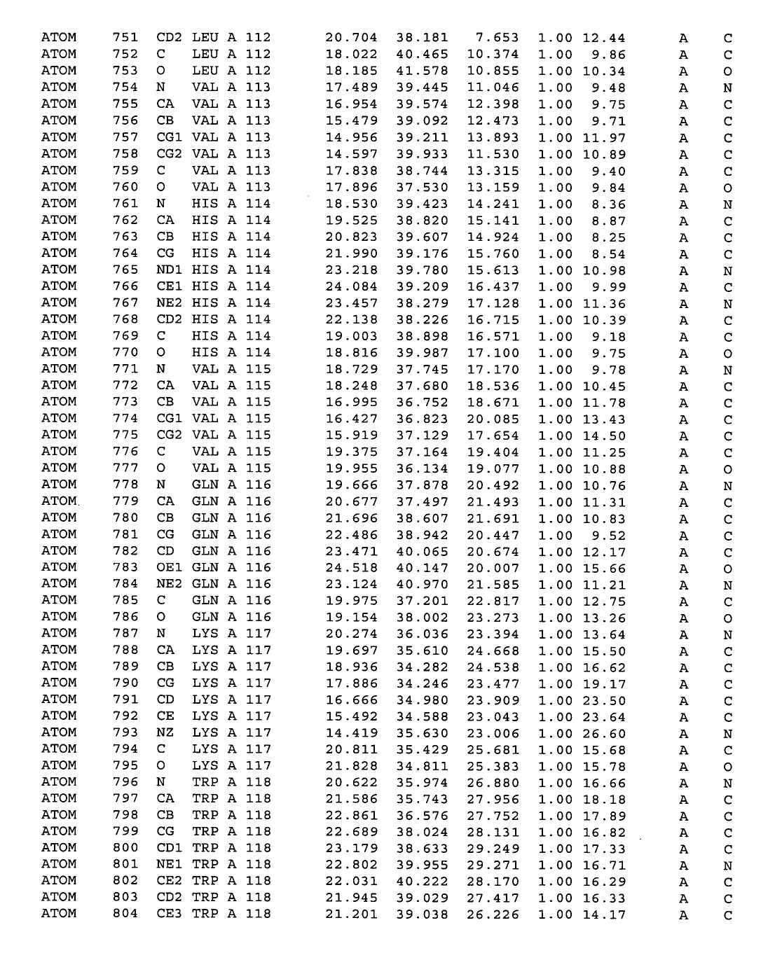

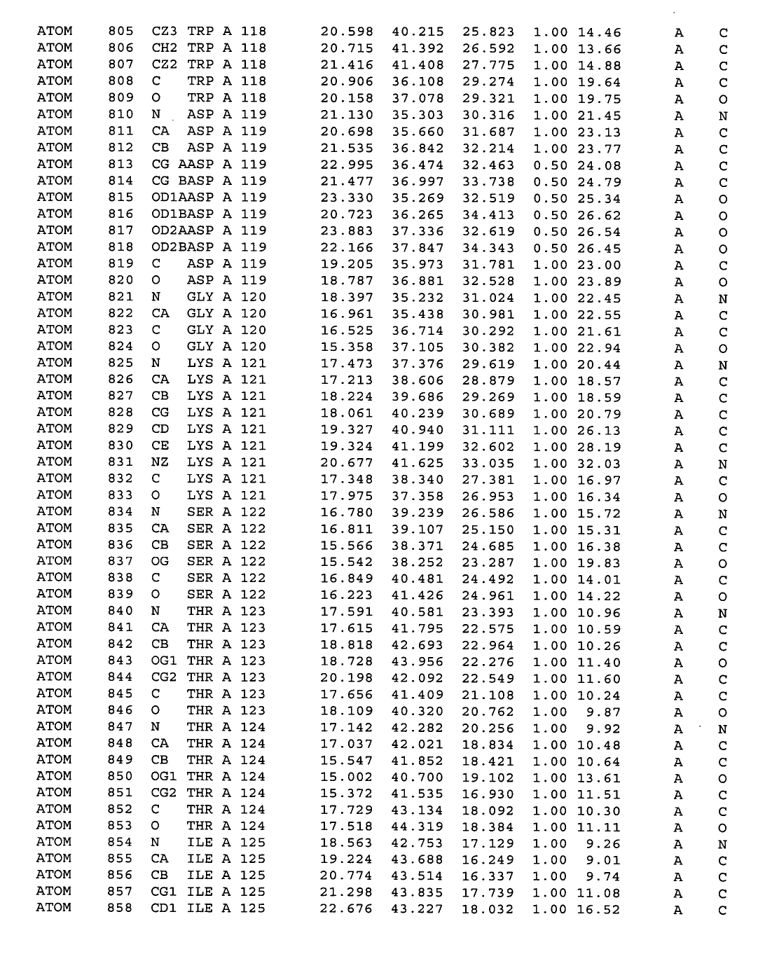

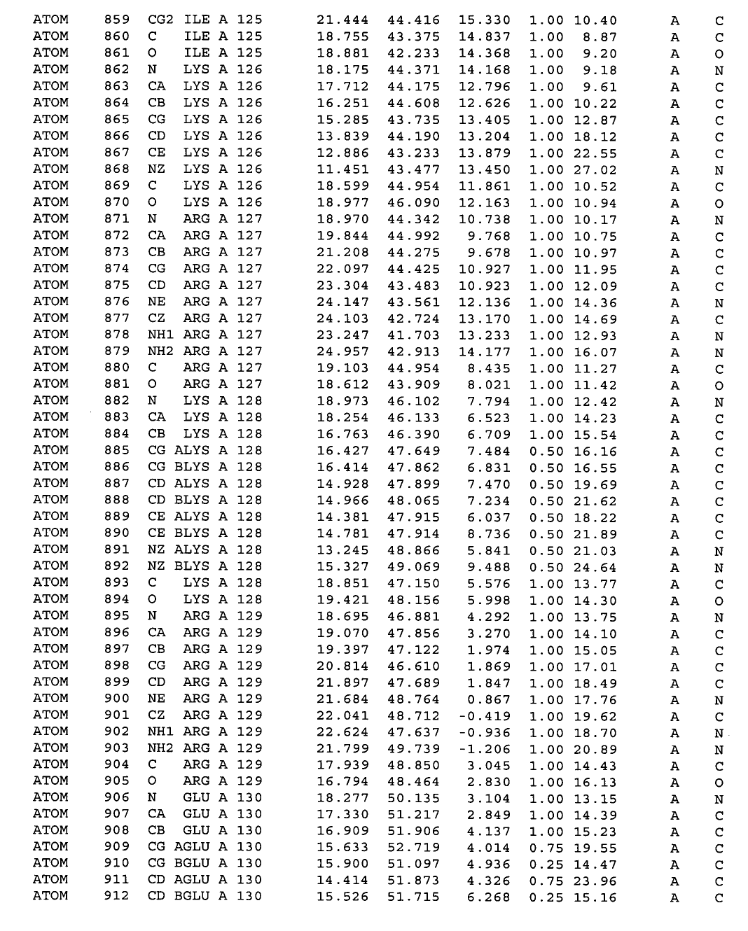

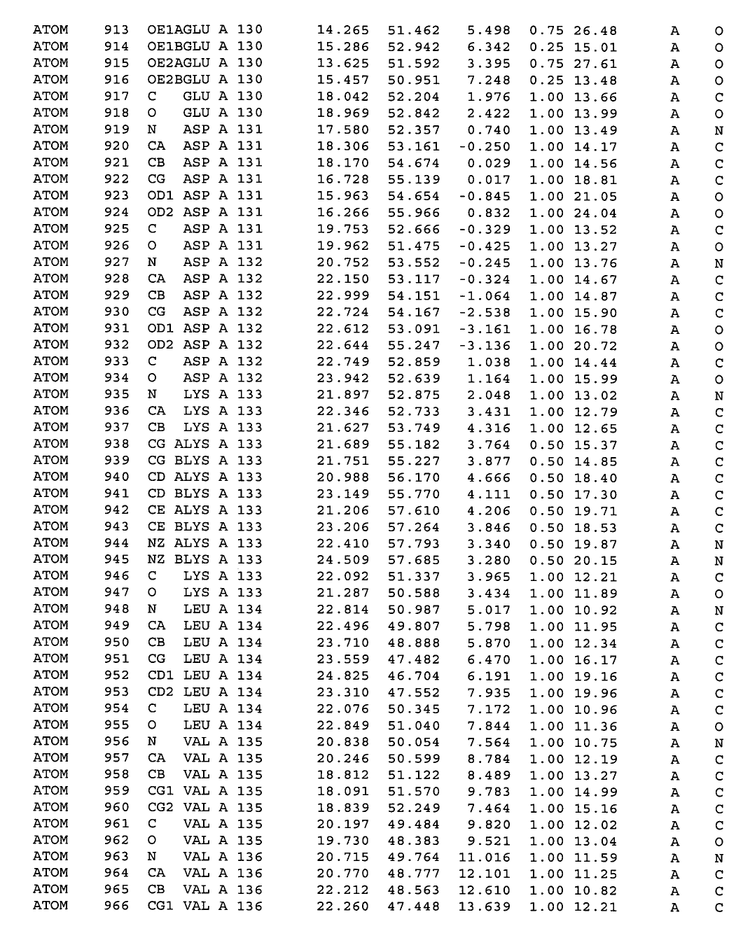

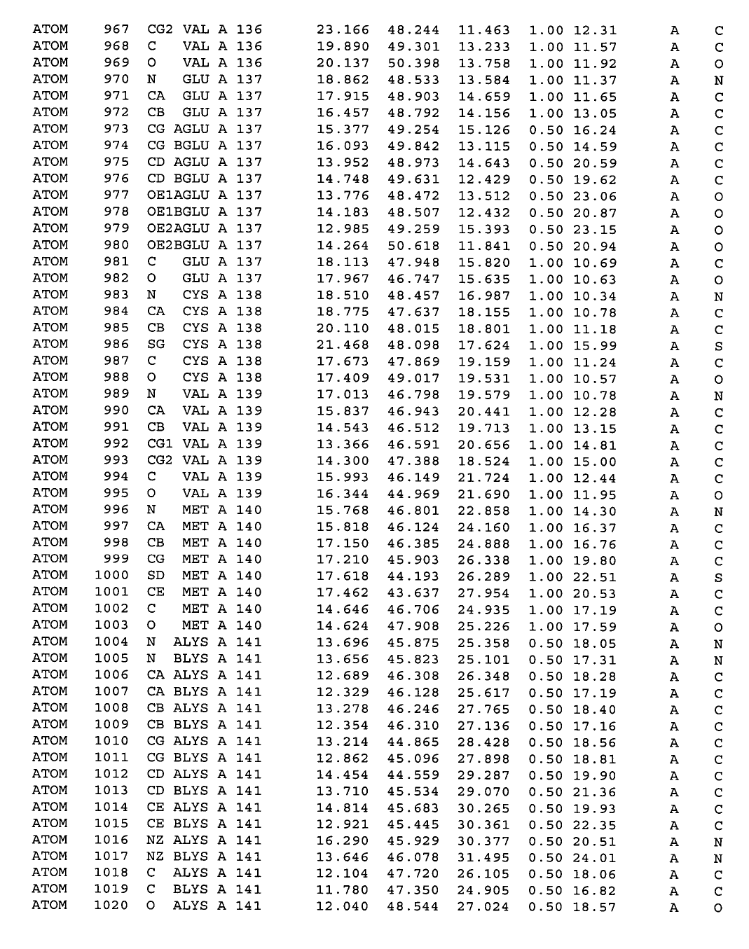

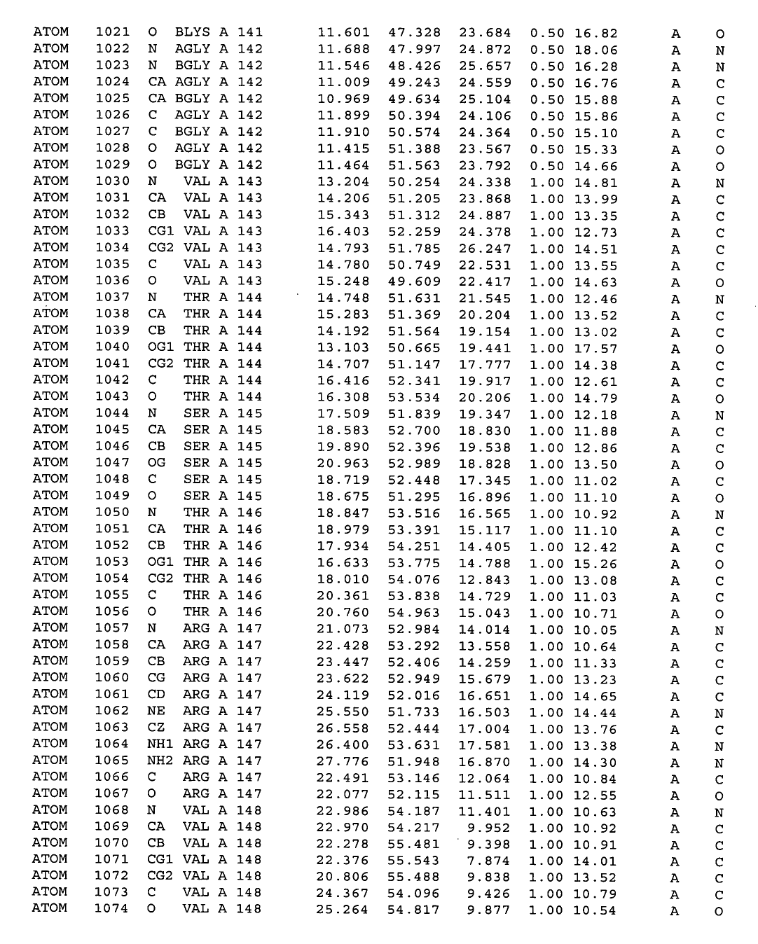

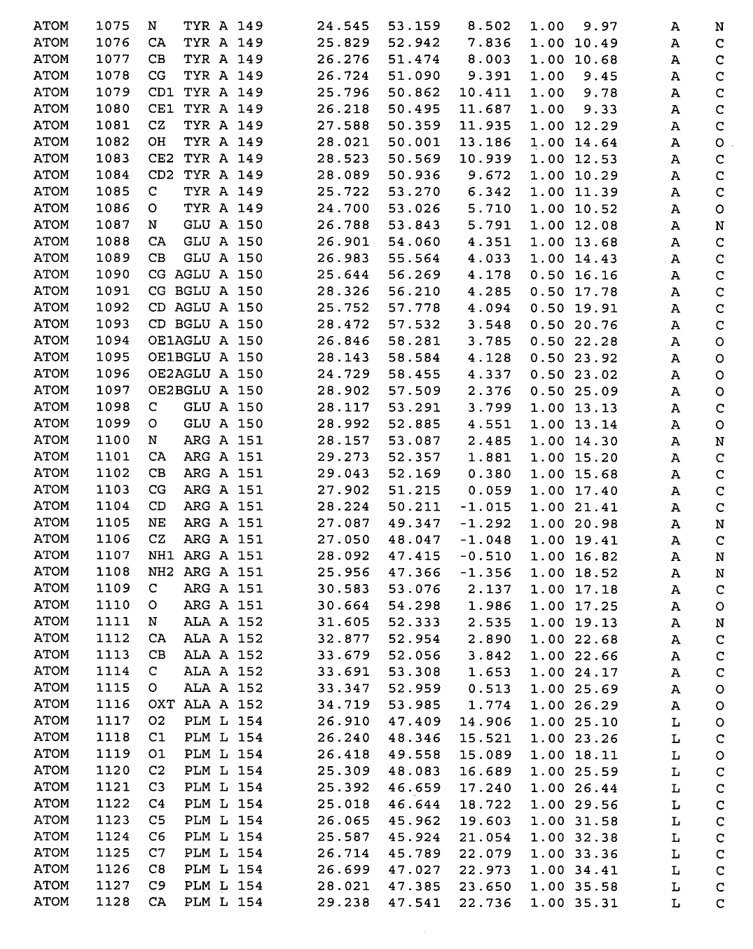

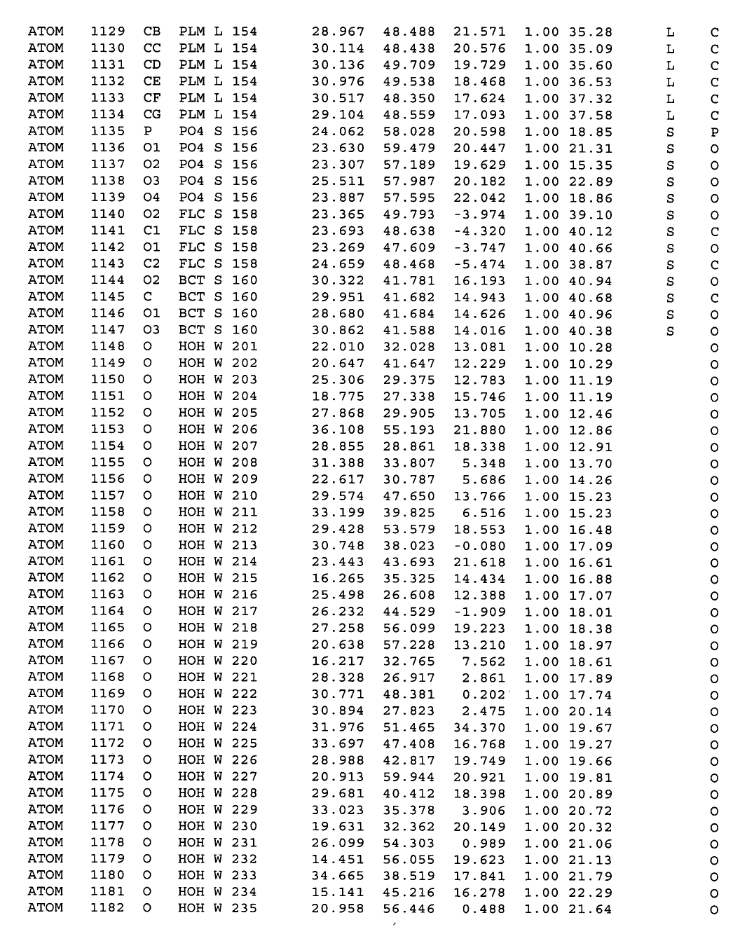

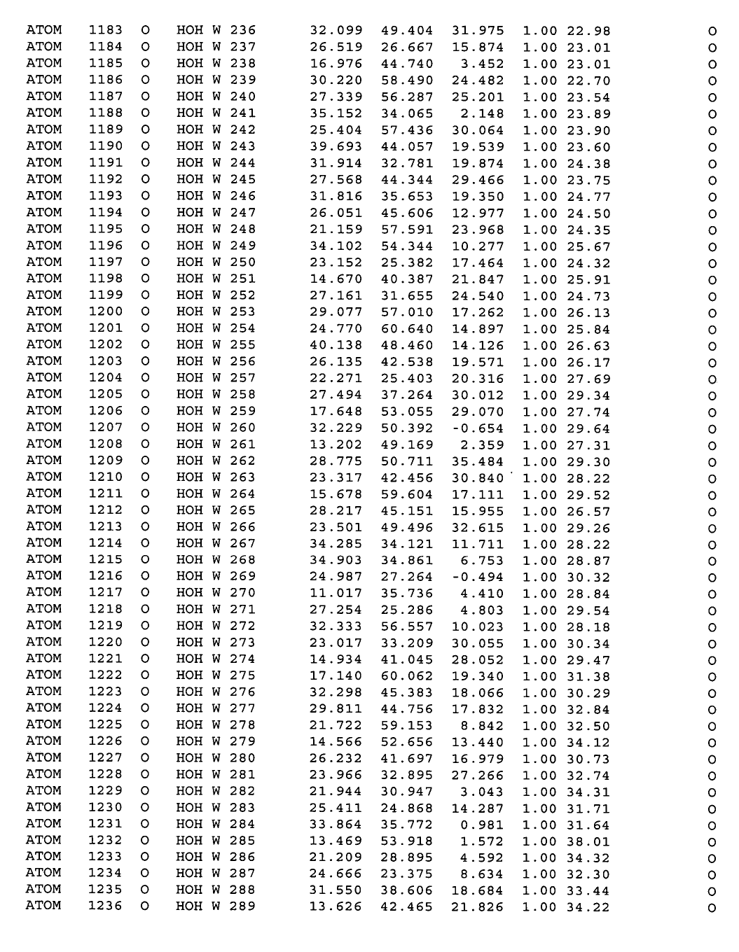

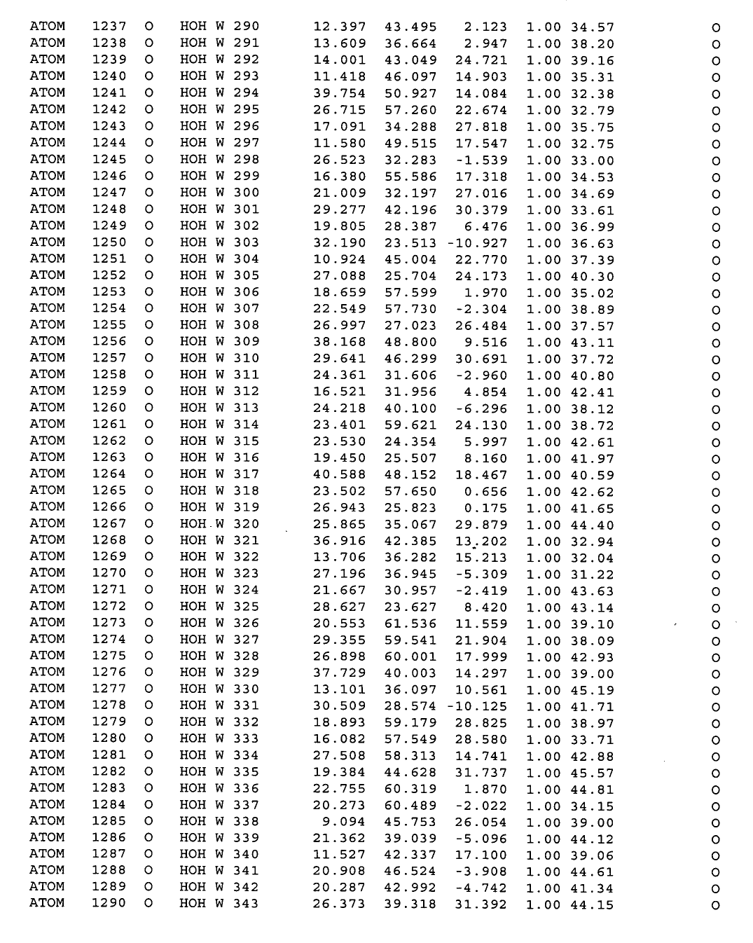

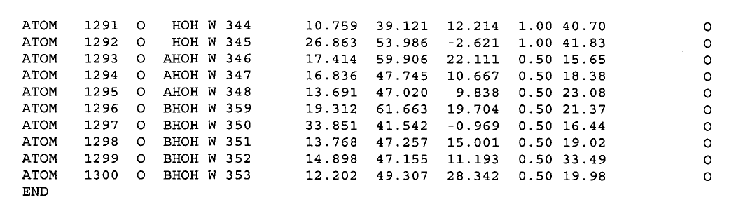

Those of skill in the art understand that a set of structure coordinates determined by X-ray crystallography is not without standard deviation. For the purpose of this invention, any set of structure coordinates for lipid binding protein or lipid binding protein homologues or lipid binding protein mutants that have a root mean square deviation of protein backbone atoms (N, C_{α}, C and O) of less than 0.75 Å when superimposed, using backbone atoms, on the structure coordinates listed in FIG. 3 shall be considered identical.

The term “unit cell” refers to a basic parallelepiped shaped block. The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal.

The term “molecular replacement” refers to a method that involves generating a preliminary model of a lipid binding protein crystal whose structure coordinates are unknown, by orienting and positioning a molecule whose structure coordinates are known (e.g., variant lipid binding protein coordinates from FIG. 3) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subjected to any of the several forms of refinement to provide a final, accurate structure of the unknown crystal. See, for example, Lattman, Methods in Enzymology, 115:55-77 (1985); M. G. Rossmann, ed., “The Molecular Replacement Method”, Int. Sci. Rev. Ser., No. 13, Gordon & Breach, New York, (1972). Using the structure coordinates of lipid binding protein provided by this invention, molecular replacement may be used to determine the structure coordinates of a crystalline mutant or homologue of lipid binding protein or of a different crystal form of lipid binding protein.

“Atom type” in, for example, FIG. 3, refers to the element whose coordinates are measured. The first letter in the column in FIG. 3 defines the element.

“X, Y, Z” crystallographically define the atomic position of the element measured.

“B” is a thermal factor that measures movement of the atom around its atomic center.

Atomic coordinates for lipid binding protein according to FIG. 3 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, and any combination of the above. The atomic coordinates of FIG. 3 correspond to the lipid binding protein polypeptide chain, and to several molecules bound thereto, including bicarbonate ion, phosphate ion, and a plurality of water molecules.

### MATERIALS AND METHODS

### Expression and Purification of aP2

The protein was over-expressed in E. coli as a His-tagged version, with amino acids 1-21 of SEQ ID NO:1 being the HIS-tag portion, and amino acids 22-152 being the human aP2 protein portion. His-tagged aP2 was isolated by nickel affinity chromatography with Ni-NTA Superflow resin (QIAGEN GmbH, Hilden, Germany). Buffer compositions were as follows: Lysis Buffer is 50 mM NaH₂PO₄, pH 8.0, 300 mM NaCl, 10 mM imidazole; Wash Buffer is 50 mM NaH₂PO₄, pH 8.0, 300 mM NaCl, 20 mM imidazole; Elution Buffer is 50 mM NaH₂PO₄, pH 8.0, 300 mM NaCl, 250 mM imidazole; Final Buffer is 50 mM NaH₂PO₄, pH 7.5, 50 mM NaCl.

5.2 g of cell paste was brought up to a final weight of 26.5 g with Lysis Buffer. One tablet of Complete™ EDTA-free protease inhibitor cocktail (Roche Diagnostics Gmbh, Basel, Switzerland) was added. Cells were resuspended by stirring, at 4° C. The suspension was subjected to two freeze/thaw cycles (−80° C. for one hour and thawing at room temperature) and stored overnight at 4° C. The suspension was sonicated until complete lysis was obtained. The lysate was centrifuged at 14,000×g for 50 min. The cleared supernatant was applied to a column, with 11 ml of packed Ni-NTA superflow resin, at 1.0 ml/min. The column was washed with Wash Buffer and step eluted with Elution Buffer. The desired fractions were pooled and dialyzed against Final Buffer. Purity was determined by SDS-PAGE and concentration by UV₂₈₀.

### Crystallization of His-tagged aP2

Sparse matrix screening was performed on the His-tagged aP2:palmitate complex using the sitting drop vapor diffusion method. Drops consisting of 2 μl protein+2 μl reservoir solution were set up at a protein concentration of 10.9 mg/mL. Plates were incubated at 22° C. Two leads were identified, containing either 1.6M tri-sodium citrate dihydrate pH 6.5 or 20% PEG 10,000, 0.1M HEPES pH 7.5. Diffraction quality plate crystals were obtained by the hanging drop vapor diffusion method using 1.5 μl his-aP2:palmitate complex+1.5 μl reservoir containing 1.6M tri-sodium citrate dihydrate pH 6.5 after three days at 22° C.

### Data Collection

X-ray diffraction data were collected from flash-frozen crystals at 100° K. Crystals were briefly soaked in a cryoprotectant solution which consisted of 20% of ethylene glycol and 80% of the crystallization well solution. They were then introduced into a 100° K. cold nitrogen stream.

Crystals are tetragonal, belong to the space group P2₁2₁2₁, with unit cell dimensions of a=32.5 Å, b=53.9 Å, c=75.0 Å, α=90°, β=90° and γ=90°. The datasheet for structural solution was collected to 1.5 Å resolution at the IMCA beamline of Advanced Photon Sources using an CCD detector. There were a total of 90080 observations and 20751 unique reflections. The Rsym is 0.047 (0.346 in the last shell) with 4.3 fold redundancy (3.2 in the last shell). The data was 95.3% (69.3% in the last shell) complete with an average l/sigl of 31 (3.2 in the last shell). All these indicated excellent quality up to 1.5 Å resolution.

### Structure Determination by Molecular Replacement

The crystal structure of a variant human aP2 according to SEQ ID NO:1 was solved by molecular replacement using the murine aP2 structure (IRL, murine adipocyte lipid binding protein with arachidonic acid (20:4) bound (LaLonde et al., J. Biol. Chem., 269:25339-47 (1994)). The structure includes residues 17 to 152 of SEQ ID NO:1, and one molecule each of palmitic acid anion, bicarbonate anion, phosphate anion and citrate anion (the latter partially disordered). The structure also includes 153 molecules of bound water. A final resolution of 1.5 Å was achieved with an Rfactor of 0.182 and R_{free} of 0.232 using the program REFMAC of the CCP4 program suite. Clear electron density was seen for bound palmitate and bicarbonate and phosphate. Refinement statistics are in Table 1A. Space group data are in Table 1B. Final refined coordinates of human aP2 are shown in FIG. 3.

TABLE 1A

| | |
|---|---|
| Resolution | 1.5 Å |
| Reflections | 18843 |
| R_{work} | 0.182 |
| R_{free} | 0.232 |
| RMSD in bond lengths | 0.012 Å |
| RMSD in bond angles | 1.4° |
| Total atoms (non-hydrogen) | 1294 |
| Ramachandran Plot | 94.3% |
| Most favoured | |
| Ramachandran Plot | 4.9% |
| Allowed | |

TABLE 1B

| | |
|---|---|
| Spacegroup | P2₁2₁2₁ (a = 32.5, b = 53.9, c = 75.0 Å) |
| Diffraction data | 95.3% Complete |
| R_{merge} | 0.047 |
| Molecular replacement | Using murine structure (1ADL) |
| Refined R | 0.182 |
| R_{free} | 0.232 |
| Structure includes | a.a. residues 17 to 152, 1 palmitic |
| | acid, 1 bicarbonate anion, 1 |
| | phosphate anion, 1 partially |
| | disordered citrate anion and 153 |
| | water molecules |

### The Structure of Human Lipid Binding Protein

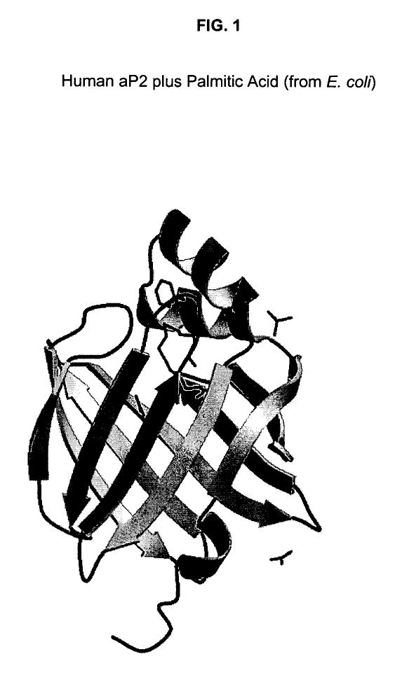

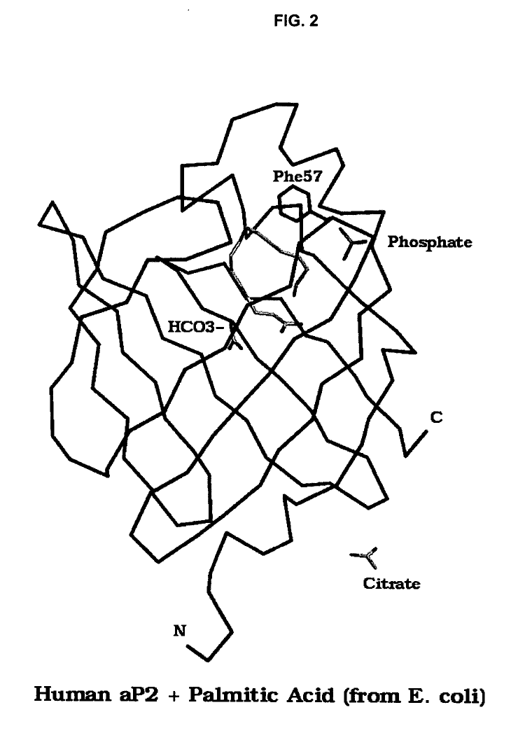

Turning to FIG. 1 and FIG. 2, the overall structure of the protein is two alpha helices that form a cap over a barrel of ten beta strands that enclose a lipid binding site.

The binding site is defined as consisting of at least one of the following residues: Phe³⁷, Tyr⁴⁰, Arg¹²⁷, Arg¹⁴⁷, and Tyr¹⁴⁹. In mutants or homologs of human lipid binding protein the numbering of amino acid residues can be normalized to the human reference sequence.

The human lipid binding protein has several structural features indicated in Table 1C below.

TABLE 1C
Lipid Binding protein Secondary Structure Assignments

| SEQ ID NO: | SEQ | TYPE |
|---|---|---|
| 2 | Gly²⁷-Glu³⁵ | S1 |
| 3 | Phe³⁷-Val⁴⁶ | H1 |
| 4 | Phe⁴⁸-Met⁵⁶ | H2 |
| 5 | Pro⁵⁹-Asn⁶⁶ | S2 |
| 6 | Val⁶⁹-Glu⁷⁵ | S3 |
| 7 | Asn⁸⁰-Phe⁸⁵ | S4 |
| 8 | Phe⁹¹-Phe⁹⁴ | S5 |
| 9 | Lys¹⁰⁰-Asp¹⁰⁸ | S6 |
| 10 | Val¹¹⁰-Trp¹¹⁷ | S7 |
| 11 | Lys¹²¹-Glu¹³⁰ | S8 |
| 12 | Lys¹³³-Met¹⁴⁰ | S9 |
| 13 | Val¹⁴³-Arg¹⁵¹ | S10 |

In Table 1C, beta-strands are labeled S1-S10 and helices are labeled H1-H2. The helices are alpha helices. Secondary structures have been calculated according to the method of Kabsch and Sander (Biopolymers, 22:2577-637 (1983)), as implemented in the program Procheck (Laskowski et al., J. Appl. Cryst., 26:283-91 (1993)). Other algorithms used to calculate secondary structure can produce slightly different assignments.

The human lipid binding protein as depicted in FIG. 1 and FIG. 2 has several notable structural features, including the following. The amino acid residues from position 27-35 are part of a beta sheet strand termed S1. H1 is an alpha helix adjacent to S1 and consists of amino acid residues from Phe³⁷ to Val⁴⁶. The amino acid residues from position 48 to 56 form an alpha helix termed H2. The amino acid residues from position 59 to 66 are part of a beta sheet strand termed S2. The amino acid residues from position 69 to 75, form S3.

Moreover, the binding protein has other notable features. The amino acids from Asn⁸⁰ to Phe⁸⁵ form a beta strand termed S4. The amino acid residues from Phe⁹¹ to Val⁹⁴ form a part of a beta sheet termed S5. The amino acid residues from Lys¹⁰⁰ to Asp¹⁰⁸ form a beta strand termed S6. The amino acids from Val¹¹⁰ to Trp¹¹⁷ form a beta strand termed S7. The amino acids from Lys¹²¹ to Gly¹³⁰ form a beta strand termed S8. The amino acid residues from Lys¹³³ to Met¹⁴⁰ form a beta strand termed S9. The amino acid residues from Val¹⁴³ to Arg¹⁵¹ form a beta strand termed S10.

The crystal structure clearly provides a description of the interaction of the amino acid residues of lipid binding protein with palmitate and the bicarbonate and phosphate ions. The Arg¹⁴⁷ has a guanidium functional group, a nitrogen atom of which interacts with a carboxylate oxygen of palmitate at a distance of about 2.7 Å. This coordination serves to lock the fatty acid into a position for the hydrophobic atoms of the lipid tail to interact with hydrophobic amino acid residues.

In this regard, the binding protein interaction with palmitate is mediated by other amino acid residues. Phe³⁷ has carbon atoms that interact with the carbon atoms of the lipid tail in palmitate, with carbon to carbon hydrophobic bond interactions having distances of about 3.7 Å

The Tyr¹⁴⁹ has a hydroxyl group having an oxygen that interacts with an oxygen atom of the carboxyl group of palmitate forming hydrogen bonds with inter-atomic distances of about 2.5 Å

One aspect of the invention relates to compositions comprising human lipid binding protein and a ligand in crystalline form. In general, the ligand can be a activator, inhibitor, or co-factor. More specifically, the ligand can be selected from the group consisting of a fatty acid, bicarbonate anion, phosphate anion, citrate anion, and any inhibitor that binds to a ligand binding site. The inhibitor can be any inhibitor of the binding protein, including a low affinity or high affinity inhibitor. In one aspect the crystal comprises ligands, or parts thereof, having atomic coordinates according to FIG. 3, or portions thereof.

In another aspect the crystalline lipid binding protein comprises amino acid residues having atomic coordinates according to FIG. 3, or a substantial portion thereof. In another aspect, the lipid binding protein comprises an amino acid sequence corresponding to residues 1 to 152 of SEQ ID NO:1.

The invention also relates to at least a first ligand binding site of lipid binding protein. The first ligand binding sites is defined by amino acid residues that interact with the typically polar or ionic head group of the ligands and, optionally, with other amino acid residues that interact with the typically hydrophobic tail of the ligand. Alternatively, the amino acid residues of the binding sites can interact indirectly with the substrate, for example, by binding to a cofactor which in turn binds to an inhibitor, or by binding to another amino acid residue which in turn binds to an inhibitor.

The first ligand binding site can be defined as comprising at least one amino acid residue selected from the group consisting of Phe³⁷, Tyr⁴⁰, Ala⁵⁴, Pro⁵⁹, Ser⁷⁴, Ala⁹⁶, Asp⁹⁷, Arg¹²⁷, Cys¹³⁸, Arg¹⁴⁷, and Tyr¹⁴⁹. In a preferred embodiment, the residues are selected from the group consisting of Phe³⁷, Tyr⁴⁰, Arg²⁷, Arg⁴⁷, and Tyr⁴⁹. In a yet more preferred embodiment, the first ligand binding site comprises at least three of these more preferred amino acid residues. In a yet still more preferred embodiment, the first ligand binding site comprises at least five of these more preferred amino acid residues.

The first ligand binding site can alternatively comprise at least about 80% of the amino acid residues selected from the group consisting of Phe³⁷, Tyr⁴⁰, Arg¹²⁷, Arg¹⁴⁷, and Tyr¹⁴⁹.

Another aspect of the invention relates to methods of designing or identifying a ligand for a lipid binding protein, the methods comprising using a three-dimensional structure including atomic coordinates of amino acid residues 37, 40, 127, 147, 149, according to FIG. 3. In a preferred embodiment, the coordinates are those of a variant human lipid binding protein, or a substantial portion thereof. The methods can include obtaining the ligand which can include synthesizing the ligand in whole or in part, purchasing the ligand, or any other suitable means.

In one aspect the invention is directed to computational models of a composition comprising a variant construct of a lipid binding protein having atomic coordinates of a variant construct of human lipid binding protein, or a portion thereof, and a computer program running on a computer addressing the atomic coordinates. The atomic coordinates can be those of FIG. 3, or a substantial portion thereof.

In another aspect, the invention is directed to methods of designing or identifying a ligand or an inhibitor of a second lipid binding protein comprising: (a) using a three-dimensional structure of a first lipid binding protein, as defined by atomic coordinates according to FIG. 3, or a substantial portion thereof; (b) identifying at least one first amino acid residue having a first peptide backbone and the amino acid residue(s) defining, in part, at least one ligand binding site; (c) employing protein alignment means to identify in the second lipid binding protein at least one second amino acid residue having a second peptide backbone that is capable of substantially aligning with the first backbone; (d) employing the three-dimensional structure to design or select the ligand for the second lipid binding protein; (e) synthesizing the ligand; and (f) contacting the ligand with the second lipid binding protein to determine binding to the second lipid binding protein; wherein the second amino acid residue differs from the first amino acid residue.

In yet another aspect, the invention is directed to computational models of an active site of an isolated lipid binding protein comprising a bicarbonate or phosphate ion cofactor and a polypeptide comprising a first arginine residue having a guanidino group having nitrogen atoms, and a tyrosine acid residue comprising an oxygen atom forming a hydroxyl functional group, wherein the oxygen atom and at least one nitrogen atom of the guanidino group of the arginine residue coordinates with a lipid head group; and a second arginine residue in a beta strand comprising the sequence Lys Ser Thr Thr Ile Lys Arg Lys Arg Glu (SEQ ID NO:11). The second Arg has at least one nitrogen atom capable of coordinating an atom of a ligand. The tyrosine residue, first arginine residue, and second arginine residue form at least a part of a binding site of a lipid binding protein.

The invention is also directed to computational models of an active site comprising a representation of the active site of lipid binding protein by a computer program capable of running on a computer.

In one aspect, the invention is directed to computational models of a composition comprising a lipid binding protein having at least twelve of the atomic coordinates of human lipid binding protein and a computer program running on a computer addressing the atomic coordinates. Preferably, the model comprises at least twenty-four, more preferably at least 36 atomic coordinates, and most preferably at least 48 atomic coordinates.

The computational model can further comprise an amino acid residue sequence (SEQ ID NO:13), the sequence having an arginine having at least one nitrogen atom, and a ligand comprising at least one oxygen atom, wherein the at least one nitrogen atom abuts the oxygen atom by about 2.4 to 3.5 Å.

In another embodiment, the computational model can further comprise an amino acid residue sequence (SEQ ID NO:13), the sequence having a tyrosine having an oxygen atom, and a ligand comprising at least one oxygen atom, wherein the tyrosine oxygen atom abuts the other oxygen by about 2.4 to 3.5 Å.

In yet another embodiment, the computational model can alternatively further comprise an amino acid residue sequence (SEQ ID NO:11), the sequence having Arg having at least one nitrogen atom, and a ligand comprising at least one oxygen atom, wherein the nitrogen atom abuts the oxygen atom by about 2.4 to 3.5 Å.

### Design of Lipid Binding Protein Inhibitors

One of skill in the art can use any method to screen chemical moieties for the ability to associate with a lipid binding protein or with the bicarbonate, phosphate or citrate binding sites that comprise other sites of the lipid binding protein. Visual inspection of a model of the ligand binding sites based on the lipid binding protein coordinates in FIG. 3 can lead to candidate chemical entities. Selected chemical moieties can then be positioned in orientations within one of the ligand binding sites of lipid binding protein. Positioning can be accomplished using software such as QUANTA and SYBYL and is useful for changing the positions of chemical entities. Then standard molecular mechanics forcefields, such as CHARMM and AMBER can be used to minimize the energy and molecular kinetics of binding.

Other computer programs useful in selecting chemical moieties include:

1. DOCK (Kuntz et al., “A Geometric Approach to Macromolecule-Ligand Interactions”, J. Mol. Biol., 161:269-88 (1982)). DOCK is available from University of California, San Francisco, Calif.

2. GRID (Goodford, “A Computational Procedure for Determining Energetically Favorable Binding Sites on Biologically Important Macromolecules”, J. Med. Chem., 28:849-57 (1985)). GRID is available from Oxford University, Oxford, UK.

3. AUTODOCK (Goodsell and Olsen, “Automated Docking of Substrates to Proteins by Simulated Annealing”, Proteins: Structure. Function, and Genetics, 8:195-202 (1990)). AUTODOCK is available from Scripps Research Institute, La Jolla, Calif.

4. MCSS (Miranker and Karplus, “Functionality Maps of Binding Sites: A Multiple Copy Simultaneous Search Method.” Proteins: Structure. Function and Genetics, 11:29-34 (1991)). MCSS is available from Molecular Simulations, Burlington, Mass.

Selected moieties can be assembled into a single compound by initial visual review of the organization of the parts to make a whole in relation to the atomic coordinates of undecaprenyl pyrophosphate synthase. Model building with software such as Quanta or Sybyl can supplement the process.

Other programs useful in building chemical moieties into a ligand or inhibitor include:

1. CAVEAT (Bartlett et al, “CAVEAT: A Program to Facilitate the Structure-Derived Design of Biologically Active Molecules”. In “Molecular Recognition in Chemical and Biological Problems”, Special Pub., Royal Chem. Soc., 78:182-96 (1989)). CAVEAT is available from the University of California, Berkeley, Calif.

2. 3D Database systems such as MACCS-3D (MDL Information Systems, San Leandro, Calif.). See also, Martin, “3D Database Searching in Drug Design”, J. Med. Chem., 35:2145-54 (1992)).

3. HOOK (available from Molecular Simulations, Burlington, Mass.).

An undecaprenyl pyrophosphate synthase inhibitor or ligand can be prepared one moiety at a time, as described. Moreover, inhibitory or other undecaprenyl pyrophosphate synthase binding compounds can be designed “de novo” using either a vacant binding site or with moieties of a known inhibitor. Computer programs that support this approach include:

1. LEGEND (Nishibata and Itai, Tetrahedron, 47:8985 (1991)). LEGEND is available from Molecular Simulations, Burlington, Mass.

2. LUDI (Bohm, “The Computer Program LUDI: A New Method for the De Novo Design of Enzyme Inhibitors”, J. Comp. Aid. Molec. Design, 6:61-78 (1992)). LUDI is available from Biosym Technologies, San Diego, Calif.

3. LeapFrog (available from Tripos Associates, St. Louis, Mo.).

Variations on molecular modeling can be useful in connection with this invention and include: Cohen et al., “Molecular Modeling Software and Methods for Medicinal Chemistry”, J. Med. Chem., 33:883-94 (1990); and Navia and Murcko, “The Use of Structural Information in Drug Design”, Current Opinions in Structural Biology, 2:202-10 (1992).

The efficiency of a model ligand binding to lipid binding protein can be evaluated and optimized by computation. For example, an effective lipid binding protein inhibitor can induce a relatively small deformation upon binding, that is, the energy in the bound and free states would be similar. Thus, in one embodiment lipid binding protein inhibitors should preferably have a deformation energy upon binding of about 8 kcal/mole or less. In the case where lipid binding protein inhibitors can bind to the binding protein in more than one conformation the deformation binding energy is the difference between the average energy of the bound conformations less the energy in free solution. Further enhancement of binding can be achieved by computational repulsive charge interaction between the ligand and the binding protein. In a similar manner, dipole-dipole interactions can be reduced. Advantageously, the net dipole-dipole and charge interactions between ligand and lipid binding protein favor binding.

Computer software useful to evaluate energies of deformation and of electrostatic repulsion and attraction include: Gaussian 92, revision C, M. J. Frisch, Gaussian, Inc., Pittsburgh, Pa. ©1992; AMBER, version 4.0, P. A. Kollman, University of California at San Francisco, ©1994; QUANTA/CHARMM, Molecular Simulations, Inc., Burlington, Mass. ©1994; and Insight II/Discover (Biosysm Technologies Inc., San Diego, Calif. ©1994). These applications can be used on suitable workstations. Other hardware systems and software packages will be known to those skilled in the art.

A model lipid binding protein-binding compound can then be modified by changing functional groups to improve binding or inhibitory properties. The modified group can be similar to the size, volume and distribution of polar and hydrophobic functional groups as the model compound or it can differ. Modified compounds can be analyzed for fit to lipid binding protein by the computer modeling methods described above.

One aspect of the invention comprises methods of identifying an inhibitor capable of binding to and inhibiting the enzymatic activity of a lipid binding protein, comprising: (a) introducing into a suitable computer program information defining the binding site of the lipid binding protein comprising first atomic coordinates of amino acids capable of binding to a substrate, wherein the program displays the three-dimensional structure thereof; (b) creating a three dimensional model of a test compound in the computer program; (c) displaying and superimposing the model of the test compound on the structure of the active site; (d) assessing whether the test compound model fits spatially into the active site; (e) incorporating the test compound in a biological binding protein activity assay; and (f) determining whether the test compound inhibits enzymatic activity in the assay.

The methods can further comprise introducing into the computer program second atomic coordinates of water molecules bound to the substrate. Thereby, the free energy of binding of the inhibitor can include displacement of bound water.

In one embodiment, the methods comprise introducing into the computer program an amino acid residue sequence of the binding protein, or portion thereof. In another embodiment, the methods further comprise introducing into the computer program atomic coordinates of at least one binding protein structural element selected from the group consisting of an alpha helix, a strand of beta sheet, and a coil.

In yet another embodiment of the methods, the lipid binding protein structural elements consist essentially of a coil and (a) a first beta sheet strand consisting of SEQ ID NO:2, or homolog thereof, and a second coil; (b) a first alpha helix consisting of SEQ ID NO:3, or homolog thereof, and a third coil; (c) a second alpha helix consisting of SEQ ID NO:4, or homolog thereof, and a fourth coil; (d) a second beta sheet strand consisting of SEQ ID NO:5, or homolog thereof, and a fifth coil; (e) a third beta strand consisting of SEQ ID NO:6, or homolog thereof, and a sixth coil; (f) a fourth beta strand consisting of SEQ ID NO:7, or homolog thereof, and a seventh coil; (g) a fifth beta sheet strand consisting of SEQ ID NO:8, or homolog thereof, and an eighth coil; (h) a sixth beta sheet strand consisting of SEQ ID NO:9, or homolog thereof, and a ninth coil; (i) a seventh beta sheet strand consisting of SEQ ID NO:10, or homolog thereof, and a tenth coil; (j) an eighth beta sheet strand consisting of SEQ ID NO:11, or homolog thereof, and an eleventh coil; (k) a ninth beta sheet strand consisting of SEQ ID NO:12, or homolog thereof, and a twelfth coil; and (l) a tenth beta sheet strand consisting of SEQ ID NO:13, or homolog thereof, and a thirteenth coil.

In still another embodiment of the methods, the second coil is connected to the first alpha helix, the third coil is connected to the second alpha helix, the fourth coil is connected to the second beta sheet strand, the fifth coil is connected to the third beta strand, the sixth coil is connected to the fourth beta strand, the seventh coil is connected to the fifth beta sheet strand, the eighth coil is connected to the sixth beta strand, the ninth coil is connected to the seventh beta strand, the tenth coil is connected to the eighth beta sheet strand, the eleventh coil is connected to the ninth beta strand, and the twelfth coil is connected to the tenth beta strand. In this description, the numerical adjectives, first, second and so forth, do not necessarily indicate a temporal or spatial order, but rather, serve merely to distinguish otherwise similarly named elements from one another.

Knowledge of the three-dimensional structure allows solution, by the method of molecular replacement, of crystal structures of lipid binding protein bound to inhibitors, and use of the method of difference Fourier analysis to determine the bound conformation of the inhibitors. Knowledge of the bound conformation then allows for the design of inhibitors with better properties, such as tighter binding.

Knowledge of the three-dimensional structure allows the user to solve, by the method of molecular replacement, the structure of lipid binding protein from any other organism.

Knowledge of the three-dimensional structure allows the user to solve, by the method of molecular replacement, the structures of lipid binding protein mutants which may be used as probes of lipid binding protein activity.

### EXAMPLES

The following non-limiting examples are presented to further illustrate the invention.

### Example 1

### Design of an Inhibitor

The atomic coordinates of the polypeptide chains of human lipid binding protein, as identified in FIG. 3, can be used in a computer to construct a three-dimensional model of the binding site. A putative inhibitor can be fit into a binding site on the protein. One such putative inhibitor is hexadecane sulfonic acid. Modifications in the putative inhibitor can be made to prepare a virtual library of structurally related compounds. A docking program can then be used to evaluate interaction of each compound with the binding protein, and to compare and rank the relative binding of the compounds to the binding protein.

Compounds that appear to have relatively high affinity for the binding protein can be obtained or synthesized and evaluated in a biochemical or biological assay. A suitable biological assay can be a measurement of growth by, for example, changes in turbidity of a bacterial suspension culture.

### Example 2

### Use of a Known Ligand of Lipid Binding Protein Activity to Identify Novel Ligands

Novel ligands capable of binding to a lipid binding protein substrate binding site can be identified by using another known ligand, for example, 1-anilino naphthalene-8-sulfonic acid.

The atomic features of the known inhibitors or substrates are introduced into a suitable computer program that has information defining the substrate binding site. Typically, the information includes atomic coordinates of those amino acids that can bind to a known binding protein substrate, such as are identified in FIG. 3. The computer program can then display the three-dimensional structure of the binding site. Then a three-dimensional model of a test compound can be created in the computer program.

A docking program can be used to dock the model of the test compound to the structure of the binding site. That is, the program fits the test molecule into the binding site, allowing for rotation of the bonds of the molecule to test the several conformation of the test molecule, and evaluates the quality of fit. Similarly, a three dimensional model of the substrate or of an inhibitor of the binding protein can be created and docking information obtained. Then the docking parameters of the test compound can be compared to those of the substrate or of the known inhibitor. The docking program can then provide an output which can rank order the association parameters of each test or comparison molecule to the binding protein.

In consequence, candidate compounds most likely to have high affinity for the binding site can be readily identified. Synthesis of the most potent test molecules, or otherwise obtaining them, can provide physical molecules for biochemical or biological analysis.

The method can optionally include introducing the atomic coordinates of those water molecules bound to the substrate, such that the coordinates are available to the computer program. Optionally, one skilled in the art can introduce into the computer program the atomic coordinates of at least one binding protein structural element. Exemplary structural elements are an alpha helix, a strand of beta sheet, and a coil. The beta strand can have the sequence shown in SEQ ID NO:13.

The method can optionally also include incorporating the test compound in a biochemical binding protein lipid transport assay for a binding protein; and then determining whether the test compound inhibits binding protein function in the assay. Suitable inhibitors can be further assessed, for example, in cell permeability studies, viability studies, and bacteremia studies, for example by biological assays.

### Example 3

### Putative Ligands for aP2

Ligands suitable for evaluation for binding to aP2 include, but are not limited to the following: hexanoate, octanoate, decanoate, dodecanoate, tetradecanoate, hexadecanoate, octadecanoate, eicosanoate, docosanoate, hexylsulfate, octylsulfate, decylsulfate, dodecylsufate, tetradecylsulfate, hexadecylsulfate, octadecylsulfate, eicosanoylsulfate, docosanoylsulfate, hexanesulfonate, octanesulfonate, decanesulfonate, dodecanesulfonate, tetradecanesulfonate, hexadecanesulfonate, octadecanesulfonate, eicosanoanesulfonate, docosanoanesulfonate, arachidonate, azelaiate, sphingosine, dihydrosphingosine, octadecatrienoate, docosenoate, glycerol-1-oleate, glycerol-1-palmitate, glycerol-2-oleate, glycerol-2-palmitate, glycerol-1-stearate, glycerol-2-stearate, glycerol-1-myristate, glycerol-2-myristate, heptadecanoate, 2-hydroxystearate, 10-hydroxystearate, lysolecithin, lysoethanol-amine, octadecadienoate, methylenehexadecanate, lysophosphatidyl inositol, tetracosenoate, octadecenoate, hexadecenoate, nonanoate, pentadecylate, sphingosine sulfate, methyldodecanoate, methyltetradecanoate, methylhexadecanoate, methyloctadecanoate, and salts and acid forms thereof.

### Example 4

### aP2 Activity Assay: The 1,8-ANS Assay

This assay is designed to identify novel inhibitors of aP2. It is further utilized to determine inhibition constant (Ki) values for compounds that exhibit >30% inhibition in a preliminary single point screen. The assay endpoint is a reduction in fluorescence signal obtained at wavelengths of 369EX/465EM.

The 1-anilinonaphthalene 8-sulfonic acid (1,8-ANS) assay is a fluorescence assay, based on previously published data by Kane and Bernlohr (Anal. Biochem., 233:197-204 (1996)). It is designed to screen for novel compounds that will inhibit fatty acid binding to aP2. 1,8-ANS binding within the pocket of aP2 generates a fluorescence signal. The unbound compound does not produce a signal, such that upon displacement of the 1,8-ANS by an inhibitory compound, the signal is diminished. Compounds are screened in triplicate at a concentration of 10 μM. Compounds exhibiting >30% inhibition are further analyzed by performing a concentration gradient to determine a Ki value.

Solution and Buffers: Assay buffer is 50 mM NaH₂PO₄ (Aldrich 22,352-2) and 1% ethanol (Pharmco 64-17-5), pH 7.4, stored at room temperature. DMSO is dimethyl sulphoxide Hybri-max (Sigma, D2650) stored at room temperature. 1,8-ANS is prepared by making up a 40 mM stock solution of 1-anilinonaphthalene 8-sulfonic acid (Molecular Probes, A-47) in DMSO and storing it at room temperature, protected from light. For a working solution, dilute the 1,8-ANS stock solution in assay buffer. Only generate a working solution of 1,8-ANS immediately prior to running an assay. Homogenously lipidated recombinant aP2 is prepared as described infra, and stored at 4° C. in 50 mM PO_{4,50} mM NaCl, pH 7.5, 1 mM EDTA, and 5 mM DTT.

The following items are used: 96-well ½ area black plates (Costar Cat# 3694); Microcentrifuge tubes, 1.7 mL pre-lubricated (Costar Cat# 3207); and SpectraMax Gemini Fluorescence Plate Reader (Molecular Devices).

Compounds are obtained from the sample bank and used to make stock solutions of 25-50 mM in 100% DMSO. This stock solution is used to prepare the dilutions for either single point screening of compounds (typically run in triplicate at a final concentration of 10 μM) or 7 log concentration-response determinations (typically run in duplicate).

For each assay run, the following controls are added: “Total” wells—contain no compound, are brought up to the total volume of 250 μl with assay buffer; Oleic acid control—positive control run at 10 μM final concentration; and Compound positive control—run a known inhibitory compound at 10 μM final concentration (e.g., 3-(4,5-diphenyl-oxazol-2yl)-propionic acid or 1-(4-chloro-phenyl)-2-(4-hydroxy-6-trifluoromethyl-pyrimidin-2ylsulfanyl)-ethanone)

For each sample well, add 25 μl of 2 μM aP2 (500 nM final) to all assay wells of a 96-well ½ volume black plate. Dilute 1,8-ANS stock solution (40 mM) to 2 μM in assay buffer. Make enough working solution of 1,8-ANS to add 25 μl per assay well. Add 25 μl of 1,8-ANS working solution to each assay well. Pipette up and down 8-9 times to mix. Cover plate with aluminum foil when not working in plate. Add 50 μl of compound or controls to the desired wells. Cover plate with aluminum foil to protect from light. Incubate at room temperature for 10 min. Read on fluorescence plate reader at 369EX/465EM.

Inhibitors of aP2 will be identified by a reduction in fluorescence as compared to the “Total” controls. Compounds exhibiting a >30% inhibition are further analyzed by performing a concentration gradient to determine a Ki value. The Ki is derived from a non-linear, variable slope, regression of the concentration-response data using GraphPad Prism.

### Equivalents

While specific embodiments of the subject invention have been discussed, the above specification is illustrative and not restrictive. Many variations of the invention will become apparent to those skilled in the art upon review of this specification. The appended claims should be interpreted by reference to the claims, along with their full scope of equivalents, and the specification, along with such variations.

All publications and patents mentioned herein, including those items listed below, are hereby incorporated by reference in their entireties as if each individual publication or patent was specifically and individually indicated to be incorporated by reference. In case of conflict, the present application, including any definitions herein, will control.

We claim:

1. A composition comprising a human lipid binding protein in crystalline form, the binding protein having an amino acid sequence at least about 90% homologous to the sequence of SEQ ID NO:1.

2. The composition of claim 1 wherein the binding protein has the sequence of SEQ ID NO:1.

3. The composition of claim 1 wherein the binding protein comprises a first ligand binding site, a second ligand binding site, or both.

4. The composition of claim 2 wherein the binding protein comprises a first ligand binding site, a second ligand binding site, or both.

5. The composition of claim 3 or 4, comprising at least one ligand.

6. The composition of claim 5 wherein the binding protein is co-crystallized with said ligand.

7. The composition of claim 5 wherein the ligand is selected from the group consisting of hexanoate, octanoate, decanoate, dodecanoate, tetradecanoate, hexadecanoate, octadecanoate, eicosanoate, docosanoate, hexylsulfate, octylsulfate, decylsulfate, dodecylsufate, tetradecylsulfate, hexadecylsulfate, octadecylsulfate, eicosanoylsulfate, docosanoylsulfate, hexanesulfonate, octanesulfonate, decanesulfonate, dodecanesulfonate, tetradecanesulfonate, hexadecanesulfonate, octadecanesulfonate, eicosanoanesulfonate, docosanoanesulfonate, arachidonate, azelaiate, sphingosine, dihydrosphingosine, octadecatrienoate, docosenoate, glycerol-1-oleate, glycerol-1-palmitate, glycerol-2-oleate, glycerol-2-palmitate, glycerol-1-stearate, glycerol-2-stearate, glycerol-1-myristate, glycerol-2-myristate, heptadecanoate, 2-hydroxystearate, 10-hydroxystearate, lysolecithin, lysoethanol-amine, octadecadienoate, methylenehexadecanate, lysophosphatidyl inositol, tetracosenoate, octadecenoate, hexadecenoate, nonanoate, pentadecylate, sphingosine sulfate, methyldodecanoate, methyltetradecanoate, methylhexadecanoate, methyloctadecanoate, and salts and acid forms thereof.

8. The composition of claim 3 or 4, wherein the first ligand binding site is defined by at least one amino acid residue selected from the group consisting of Phe³⁷, Tyr⁴⁰, Arg¹²⁷, Arg¹⁴⁷, and Tyr¹⁴⁹.

9. The composition of claim 3 wherein the first ligand binding site comprises at least about 80% of the amino acid residues selected from the group consisting of Phe³⁷, Tyr⁴⁰, Arg¹²⁷, Arg¹⁴⁷ and Tyr¹⁴⁹.

10. The composition of claim 3 or 4, wherein the first ligand binding site is capable of associating with palmitate.

11. The composition of claim 1 or 2 wherein the binding protein comprises a first ligand binding site defined by amino acid residues 37, 40, 127, 147, and 149 having atoms having atomic coordinates according to FIG. 3.

12. A selenomethionine substitution human lipid binding protein in crystalline form.

13. A method of identifying a ligand for a human lipid binding protein, said method comprising:
(a) using a three-dimensional structure of the binding protein as defined by at least atomic coordinates of amino acid residues 37, 40, 127, 147 and 149 according to FIG. 3;
(b) employing the three-dimensional structure to design or select the ligand;
(c) obtaining the ligand; and
(d) contacting the ligand with the binding protein to determine binding of the ligand to the binding protein wherein the binding protein comprises an amino acid sequence at least about 90% homologous to SEQ ID NO:1.

14. The method of claim 13 further comprising:
(e) identifying chemical entities or fragments thereof capable of binding to the binding protein; and
(f) assembling the identified chemical entities or fragments thereof into a single molecule to provide the structure of the ligand.

15. The method of claim 13 wherein the ligand is an inhibitor.

16. The method of claim 13 wherein the ligand is designed de novo.

17. The method of claim 13 wherein the ligand is designed from a known inhibitor.

18. The method of claim 13 further comprising using the atomic coordinates, or a portion thereof, of a ligand bound to the binding protein.

19. The method of claim 13 wherein the ligand is designed to form a hydrogen bond with at least one amino acid residue selected from the group consisting of Arg¹²⁷, Arg¹⁴⁷, and Tyr¹⁴⁹.

20. The method of claim 13 wherein the ligand is designed to form a hydrophobic bond with at least one amino acid residue selected from the group consisting of Phe³⁷ and Tyr⁴⁰.

21. The method of claim 13 wherein (c) precedes (b).

22. A method for identifying an inhibitor of a mutant lipid binding protein, the method comprising:
(a) using a three-dimensional structure of lipid binding protein as defined by atomic coordinates of lipid binding protein according to FIG. 3;
(b) replacing one or more lipid binding protein amino acids selected from 37, 40, 54, 59, 74, 96, 97, 127, 138, 147, and 149 of SEQ ID NO:1 in the three-dimensional structure with a different naturally occurring amino acid, thereby forming a mutant lipid binding protein;
(c) employing the three-dimensional structure to design or select the inhibitor; and
(d) contacting the inhibitor with the mutant lipid binding protein or the lipid binding protein in the presence of a natural ligand to test the ability of the inhibitor to inhibit the lipid binding protein or the mutant lipid binding protein.

23. The method of claim 22 wherein the inhibitor is selected from a database.

24. The method of claim 22 wherein the inhibitor is designed de novo.

25. The method of claim 22 wherein the inhibitor is designed from a known inhibitor.

26. The method of claim 22 wherein step (c) comprises the substeps:
(i) identifying chemical entities or fragments thereof capable of associating with the mutant lipid binding protein; and
(ii) assembling the identified chemical entities or fragments thereof into a single molecule to provide the structure of the inhibitor.

27. A method of identifying a ligand capable of binding to a lipid binding protein lipid binding site, said method comprising:
(a) introducing into a suitable computer program information defining the binding site comprising first atomic coordinates of amino acids capable of binding to a lipid, wherein the program displays the three-dimensional structure of the binding site;
(b) creating a three dimensional model of a test compound in the computer program;
(c) docking the model of the test compound to the structure of the binding site;
(d) creating a second three dimensional model of the ligand or an inhibitor of the binding protein and docking the second model thereto; and
(e) comparing the docking of the test compound and of the ligand or the inhibitor of the binding protein to provide an output of the program.

28. The method of claim 27 further comprising introducing into the computer program second atomic coordinates of water molecules bound to the ligand.

29. The method of claim 27 further comprising introducing into the computer program third atomic coordinates of at least one binding protein structural element selected from the group consisting of an alpha helix, a strand of beta sheet, and a coil.

30. The method of claim 27 further comprising:
(f) incorporating the test compound into a biological or biochemical binding protein activity assay; and
(g) determining whether the test compound inhibits binding protein activity in the assay.

31. A method for identifying an inhibitor for a lipid binding protein, comprising:
(a) using a three-dimensional structure of the binding protein as defined by atomic coordinates of the binding protein according to FIG. 3;
(b) employing said three-dimensional structure to design or select the inhibitor; and
(c) contacting the inhibitor with the binding protein in the presence of a natural ligand to determine the ability of the inhibitor to inhibit the binding protein.

32. The method of claim 31 wherein the inhibitor is designed de novo.

33. The method of claim 31 wherein the inhibitor is designed from a known inhibitor.

34. The method of claim 31 wherein step (b) comprises the substeps:
(i) identifying chemical entities or fragments thereof capable of associating with the binding protein; and
(ii) assembling the identified chemical entities of fragments into a single molecule to provide the structure of the inhibitor.

35. The method of claim 34 wherein the inhibitor is designed de novo.

36. The method of claim 34 wherein the inhibitor is designed from a known inhibitor.

37. A method for solving a crystal form comprising using atomic coordinates of a human lipid binding protein crystal or portions thereof, to solve a crystal form of a mutant, homolog or co-complex of the lipid binding protein by molecular replacement.

38. The method of claim 37 comprising using atomic coordinates of a ligand bound to the lipid binding protein.

39. A machine-readable data storage medium comprising a data storage material encoded with machine-readable data comprising atomic coordinates comprising amino acid residues 37, 40, 127, 147, and 149 according to FIG. 3.

40. The machine-readable data storage medium of claim 39 wherein the machine-readable data comprise the three-dimensional structure of human lipid binding protein.

41. A computer-implemented tool for design of a drug, comprising:
(a) a three-dimensional structure of a lipid binding protein as defined by atomic coordinates of a human lipid binding protein having at least one ligand binding site;
(b) a model of a chemical entity; and
(c) a computer program addressing the coordinates and capable of modeling the chemical entity in the ligand binding site to produce an output wherein the binding protein comprises an amino acid sequence at least about 90% homologous to SEQ ID NO:1.

42. The tool of claim 41, wherein said atomic coordinates are essentially as described in FIG. 3.

43. A computer for producing a three-dimensional representation of a lipid binding protein ligand binding site comprising:
(a) a machine-readable data storage medium comprising a data storage material encoded with machine-readable data comprising the atomic coordinates comprising the amino acid residues 37, 40, 127, 147, and 149 according to FIG. 3;
(b) a working memory for storing instructions for processing the machine-readable data;
(c) a central-processing unit coupled to the working memory and to the machine-readable data storage medium for processing the machine readable data into the three-dimensional representation; and
(d) a display coupled to the central-processing unit for displaying the three-dimensional representation.

44. The computer of claim 43 wherein the computer produces a three-dimensional representation of the ligand binding site of a lipid binding protein; and wherein the machine-readable data comprises the atomic coordinates of the ligand binding site.
